(19) United States
(12) Patent Application Publication
(10) Pub. No.: US 2006/0002875 A1
(43) Pub. Date: Jan. 5, 2006

(54) Diffractive colorants for cosmetics

(76) Inventors: Holger Winkler, Darmstadt (DE); Stefan Horstmann, Heppenheim (DE); Christoph Schmidt, Kriftel (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/159,413

(22) Filed: Jun. 23, 2005

(30) Foreign Application Priority Data
Jul. 1, 2004 (DE) 102004032120.5

(51) Int. Cl. A61K 8/81

(52) U.S. Cl. 424/63

(57) ABSTRACT

The invention relates to the use of diffractive colorants in cosmetics, to compositions comprising diffractive colorants, and to processes for the preparation of the compositions and to the use thereof.

The invention relates to the use of diffractive colorants in cosmetics, to compositions comprising diffractive colorants, and to processes for the preparation of the compositions and to the use thereof.

In the area of decorative cosmetics and also the design of care or cleansing compositions with the appearance of high value, there is constant demand for novel effect materials. Besides dyes, standard commercial effect colorants are currently, for example, interference pigments.

It has now been found that diffractive colorants are also eminently suitable for use in cosmetics.

The present invention therefore relates firstly to the use of diffractive colorants in cosmetics.

In one variant of the invention, the diffractive colorants are employed for decorative purposes in topical application. In a further variant, the diffractive colorants are employed for decorative purposes in compositions for topical application. Topical application can take place here to skin, hair or nails, with application of the diffractive colorants to nails being particularly preferred in accordance with the invention.

Preferred diffractive colorants here can be particles, in particular flakes or spheres, films or aggregates.

For the purposes of the present invention, diffractive colorants are materials which have three-dimensional photonic structures. The term three-dimensional photonic structures is generally taken to mean systems which have a regular, three-dimensional modulation of the dielectric constants (and thus also of the refractive index). If the periodic modulation length corresponds approximately to the wavelength of (visible) light, the structure interacts with the light in the manner of a three-dimensional diffraction grating, which is evident from angle-dependent colour phenomena.

It is therefore preferred in accordance with the invention for the diffractive colorants to essentially consist, in the proportion essential for the optical properties, of arrangements of spherical particles or cavities having an essentially monodisperse size distribution.

An example thereof is the naturally occurring precious stone opal, which consists of closest packed silicon dioxide spheres and cavities in between which are filled with air, silica gel or water.

Natural precious opals are built up from domains consisting of monodisperse, closest-packed and therefore regularly arranged silica-gel spheres having diameters of 150-400 nm. The colour play of these opals is created by Bragg-like scattering of the incident light at the lattice planes of the domains arranged in a crystal-like manner.

Corresponding arrangements of spheres can of course also be obtained with spheres comprising other materials. Suitable spheres are described below in the paragraphs on cores for core/shell particles. All the sphere materials described as possible cores are suitable for regular arrangement in diffractive colorants.

Sphere aggregates of this type are, in particular, diffractive colorants preferably to be employed in accordance with the invention if the aggregates are mechanically stabilised. This can take place through linking of the spheres or introduction of a highly viscous matrix between the spheres. The term highly viscous materials here is taken to mean, in particular, viscous, wax-like or solid, plastic or elastic to brittle materials.

For example, sphere aggregates can be embedded in a wax-like matrix. A suitable wax-like matrix is formed, for example, by fats, waxes and other natural and synthetic fatty bodies, preferably esters of fatty acids with alcohols having a low C number, for example with isopropanol, propylene glycol or glycerol, or esters of fatty alcohols with alkanoic acids having a low C number or with fatty acids. Particularly suitable substances for the wax-like matrix include paraffins, beeswax, candelilla wax, carnauba wax, silicone waxes and microcrystalline waxes (such as, for example, ozokerite and ceresin) and in particular high-melting waxes, such as shea butter, lanolin (in particular hydrogenated grades) and myristyl myristate.

The present application furthermore relates to corresponding diffractive colorants and to a process for the preparation thereof, in which, in a first step, three-dimensional sphere packing is produced, and, in a second step, the sphere aggregates are impregnated with a melt of a material which is wax-like at room temperature. For example, regular sphere aggregates can be produced by simple sedimentation or more economically from a dispersion by filtration. Processing and in particular comminution steps which are conventional in the case of waxes then enable any desired particles, such as flakes or spheres, to be produced from the wax-stabilised sphere aggregates.

If the wax is selected in such a way that its melting point is in the range between room temperature and the temperature of the skin surface, the resultant diffractive colorant is particularly suitable for use for decorative purposes in compositions for topical application. If the composition remains on the skin for an extended time, the wax melts, the spheres lose their regular arrangement, and the optical effect of the diffractive colorant disappears. Waxes which are particularly suitable for this purpose are the above-mentioned skin-melting waxes.

Diffractive colorants of this type can then also be employed particularly advantageously for control of the application of care or protective compositions, such as day creams or sunscreens or insect-protection compositions.

The present application therefore furthermore relates to a corresponding process for assessment of the application of compositions for topical application, which is characterised in that diffractive colorants which are incorporated into the composition are applied to the skin, and it is estimated from the visible effect of the diffractive colorants to which parts of the skin application has already taken place and/or whether sufficient composition has been applied and/or when re-application has become necessary.

Further suitable stabilisation methods for sphere aggregates are known from the literature. U.S. Pat. No. 4,703,020 describes a process for the preparation of a decorative material which consists of amorphous silica beads which are arranged in a three-dimensional manner, where zirconium oxide or zirconium hydroxide is located in the interstices between the beads. The beads have a diameter of 150-400 nm. The preparation here is carried out in two steps. In a first step, silicon dioxide beads are allowed to sediment from an aqueous suspension. The resultant mass is then dried in air and subsequently calcined at 800° C. The calcined material is introduced into the solution of a zirconium alkoxide in a second step, with the alkoxide penetrating into the interstices between the cores and zirconium oxide being precipitated by hydrolysis. This material is subsequently calcined at 1000-1300° C. This diffractive colorant is also suitable for the use according to the invention.

EP-A-1285031 discloses particles having an opalescent effect which have a particle size in the range from 5 μm to 5000 μm, where the particles consist of monodisperse spheres having a diameter of 50 nm-2 μm with a standard deviation of less than 5% in a three-dimensional, regularly arranged structure which is closest-packed in domains and is mechanically stabilised by a physical or chemical modification. Also described is a process for the production of particles in which, in a step a), monodisperse spheres having a diameter of from 50 nm to 2 μm with a standard deviation of less than 5% are suspended in a liquid medium, b) the suspension is applied to a surface, and c) the liquid medium is removed. The stabilisation can be achieved by the following physical and chemical measures. The surface of the spheres is modified in order to achieve crosslinking of the spheres or better adhesion of the spheres to one another during the formation of the opal structure. For suspension of the spheres in a liquid medium, it is preferably possible to add a water-hydrolysable compound whose hydrolysis product precipitates on the spheres during the formation of the opal structure and effects chemical bonding of the spheres to one another. In the case of silicon dioxide spheres, tetraethyl orthosilicate is preferably added to the suspension at temperatures of from 50 to 80° C., is hydrolysed to give silicon dioxide and results in chemical bonding of the spheres to one another. Alternatively, silicon tetrachloride can also be employed for treatment of the coated surface. The particles according to the invention can also be chemically stabilised by addition of soluble silicates, for example sodium water-glass and/or polymerisable soluble aluminium compounds to the suspension. The stabilisation can also be achieved by treatment of the coated surface with soluble silicates. The particles can likewise be physically stabilised by embedding them in transparent plastics or suitable surface coatings. It is essential here for the embedding material to be transparent and to have a suitable refractive index which results in an optimum refractive-index difference. For easy processing of the embedding material, for example a surface coating, it is particularly advantageous for it to be of low viscosity and for its volume to change only very little or not at all during curing. In a possible embodiment, the embedding material or its precursor is already present in the suspension in a corresponding volume fraction. In a further embodiment of the solidification of the opal structure, the surface of the spheres is modified by means of silanes, which are then crosslinked with one another by heat or UV radiation during the formation of the opal structure. This crosslinking likewise results in solidification of the opal structure. The silanisation of monodisperse silicon dioxide spheres is described in greater detail in DE 43 16 814. The resultant diffractive colorants are suitable for use as pigment in cosmetics.

EP-A-0 955 323 describes core/shell particles whose core and shell materials are able to form a two-phase system and which are characterised in that the shell material can be formed into a film and the cores are essentially dimensionally stable under the conditions of the shell film formation, are only swellable by the shell material to a very slight extent or not at all, and have a monodisperse size distribution, where there is a difference of at least 0.001 between the refractive indices of the core material and the shell material. Furthermore, the production of the core/shell particles and the use thereof for the production of mouldings are described. The process for the production of a moulding comprises the following steps here: application of the core/shell particles to a substrate of low adhesive power, optionally evaporation or expulsion of any solvent or diluent present in the applied layer, conversion of the shell material of the core/shell particles into a liquid, soft or viscoelastic matrix phase, orientation of the cores of the core/shell particles at least to form domains having a regular structure, curing of the shell material for fixing the regular core structure, detachment of the cured film from the substrate and, if a pigment or powder is to be produced, comminution of the detached film to the desired particle size. In these core/shell particles disclosed in EP-A-0 955 323, the core “floats” in the shell matrix; a long-range order of the cores does not form in the melt, but instead merely a short-range order of the cores in domains. These particles are thereby of only limited suitability for processing by conventional methods for polymers. The present invention likewise relates to the use of these aggregates as diffractive colorants.

Patent application WO 03/25035 discloses mouldings which essentially consist of core/shell particles whose shell forms a matrix and whose core is essentially solid and has an essentially monodisperse size distribution, where the shell is preferably strongly bonded to the core via an interlayer. The refractive indices of the core material and of the shell material differ here, causing the said optical effect, preferably an opalescence. According to the patent application DE 10204338, contrast materials, such as pigments, can additionally be incorporated into mouldings of such core/shell particles. The incorporated contrast materials effect an increase in brightness, contrast and depth of the observed colour effects in these mouldings. Processing of such mouldings to give pigments is likewise described. The pigments can be prepared here, for example, by firstly producing a film from the core/shell particles, which film can optionally be cured. The film can subsequently be comminuted in a suitable manner by cutting or crushing and possibly subsequent grinding to give pigments of a suitable size. This operation can be carried out, for example, in a continuous belt process. The use of corresponding diffractive colorants in accordance with WO 03/25035 or DE 10204338 essentially built up from core/shell particles, the preparation and composition thereof also expressly belong to the disclosure content of the present patent application.

Diffractive colorants essentially consisting of core/shell particles whose shell forms a matrix and whose core is essentially solid and has an essentially monodisperse size distribution, where there is a difference between the refractive indices of the core material and of the shell material, and the matrix is brittle, are described in the earlier German patent application with the file reference DE 10357679.7.

The term brittle matrix here is taken to mean a matrix that has such great mechanical hardness that films having this matrix can be ground. In particular, a brittle matrix for the purposes of the present invention does not exhibit elastomeric properties and does not flow even under mechanical load. For the purposes of the present invention, a brittle matrix fractures under mechanical load and is thus particularly suitable for the production of particles by grinding relatively large units.

In a preferred embodiment of the present invention, the brittleness of the matrix is achieved through the matrix being formed essentially by crosslinked organic polymers.

In a likewise preferred embodiment of the present invention, the glass transition temperature TG of the matrix is above 25° C., preferably above 50° C. and particularly preferably above 70° C., so that the brittleness of the matrix in the above-mentioned sense is guaranteed under the usual use conditions of the diffractive colorants at room temperature or, for example, slightly elevated temperature caused by sunlight. The glass transition temperature here can be set through a suitable choice of polymers as shell material of the core/shell particles. A judicious choice presents the person skilled in the art with absolutely no problems. The shell material is particularly preferably homopolymeric or copolymeric poly(cyclohexyl methacrylate), polystyrene and substituted polystyrene derivatives, such as, for example, poly(iodostyrene) and poly(bromostyrene), polyacrylates and polymethacrylates having a Tg above the use temperature, polyvinyl chloride of high Tg and other vinylic polymers which arise through conversion of polyvinyl acetate, and polyacrylonitrile and styrene-acrylonitrile copolymers.

Apart from the aspects discussed, these diffractive colorants according to the invention are built up from core/shell particles as described in WO 03/25035.

In order to achieve the optical effect according to the invention, it is desirable for the core/shell particles to have a mean particle diameter in the range from about 5 nm to about 2000 nm. It may be particularly preferred here for the core/shell particles to have a mean particle diameter in the range from about 5 to 20 nm, preferably from 5 to 10 nm. In this case, the cores may be known as “quantum dots”; they exhibit the corresponding effects known from the literature. In order to achieve colour effects in the region of visible light, it is particularly advantageous for the core/shell particles to have a mean particle diameter in the range about 40-500 nm. Particular preference is given to the use of particles in the range 80-500 nm since, in particles in this size range, the reflections of various wavelengths of visible light differ significantly from one another, and thus the opalescence which is particularly important for optical effects in the visible region occurs to a particularly pronounced extent in a very wide variety of colours. However, it is also preferred in a variant of the present invention to employ multiples of this preferred particle size, which then result in reflections corresponding to the higher orders and thus in a broad colour play.

For the purposes of the invention, the term optical effect is taken to mean both effects in the visible wavelength region of light and, for example, also effects in the UV or infrared region. It has recently become customary to refer to effects of this type in general as photonic effects. All these effects are optical effects for the purposes of the present invention, where, in a preferred embodiment, the effect is opalescence in the visible region. In the sense of a conventional definition of the term, the diffractive colorants according to the invention are photonic crystals (cf. Nachrichten aus der Chemie; 49 (9) September 2001; pp. 1018-1025).

It is particularly preferred in accordance with the invention for the core of the core/shell particles to consist of a material which is either not flowable or becomes flowable at a temperature above the melting point of the shell material. This can be achieved through the use of polymeric materials having a correspondingly high glass transition temperature (T_{g}), preferably crosslinked polymers, or through the use of inorganic core materials. The suitable materials in detail are described below.

A further crucial factor for the intensity of the observed effects is the difference between the refractive indices of core and shell. Diffractive colorants according to the invention preferably have a difference between the refractive indices of the core material and of the shell material of at least 0.001, preferably at least 0.01 and particularly preferably at least 0.1. If the diffractive colorants according to the invention are intended to exhibit industrially usable photonic effects, refractive index differences of at least 1.5 are preferred.

In a particular embodiment of the invention, further nanoparticles are incorporated into the matrix phase of the diffractive colorants in addition to the cores of the core/shell particles. These particles are selected with respect to their particle size in such a way that they fit into the cavities of the sphere packing of the cores and thus cause only little change in the arrangement of the cores. Through specific selection of corresponding materials and/or the particle size, it is firstly possible to modify the optical effects of the diffractive colorants, for example to increase the intensity. Secondly, it is possible through incorporation of suitable “quantum dots” to functionalise the matrix correspondingly. Preferred materials are inorganic nanoparticles, in particular nanoparticles of metals or of II-VI or III-V semiconductors or of materials which influence the magnetic properties of the materials. Examples of preferred nanoparticles are gold, zinc sulfide, haematite or gallium arsenide.

The precise mechanism which results in the uniform orientation of the core/shell particles in the diffractive colorants according to the invention was hitherto unknown. However, it has been found that action of force is essential for the formation of the far-reaching order. It is assumed that the elasticity of the shell material under the processing conditions is crucial for the ordering process. The chain ends of the shell polymers generally attempt to adopt a coiled shape. If two particles come too close, the coils are compressed in accordance with the model concept, and repellent forces arise. Since the shell-polymer chains of different particles also interact with one another, the polymer chains are stretched in accordance with the model if two particles move away from one another. Due to the attempts by the shell-polymer chains to re-adopt a coiled shape, a force arises which pulls the particles closer together again. In accordance with the model concept, the far-reaching order of the particles in the diffractive colorant is caused by the interaction of these forces.

Particularly suitable core/shell particles for the preparation of diffractive colorants according to the invention have proven to be those whose shell is bonded to the core via an interlayer.

In a preferred embodiment, the shell of these core/shell particles consists of organic polymers, which are preferably grafted onto the core via an at least partially crosslinked interlayer.

The core can consist of a very wide variety of materials. The essential factor according to the invention is, as already stated, that a refractive-index difference to the shell exists and the core remains solid under the processing conditions.

It is furthermore particularly preferred in a variant of the invention for the core to consist of an organic polymer, which is preferably crosslinked.

In another, likewise preferred variant of the invention, the core consists of an inorganic material, preferably a metal or semimetal or a metal chalcogenide or metal pnictide. For the purposes of the present invention, chalcogenides are taken to mean compounds in which an element from group 16 of the Periodic Table of the Elements is the electronegative bonding partner; pnictides are taken to mean those in which an element from group 15 of the Periodic Table of the Elements is the electronegative bonding partner.

Preferred cores consist of metal chalcogenides, preferably metal oxides, or metal pnictides, preferably nitrides or phosphides. Metals in the sense of these terms are all elements which can occur as electropositive partner compared with the counterions, such as the classical metals of the subgroups, or the main-group metals from the first and second main groups, but also all elements from the third main group, as well as silicon, germanium, tin, lead, phosphorus, arsenic, antimony and bismuth. The preferred metal chalcogenides and metal pnictides include, in particular, silicon dioxide, aluminium oxide, zirconium oxide, iron oxides, titanium dioxide, cerium dioxide, gallium nitride, boron nitride, aluminium nitride, silicon nitride and phosphorus nitride or mixtures thereof.

The starting materials employed for the production of the core/shell particles according to the invention in a variant of the present invention are preferably monodisperse cores of silicon dioxide, which can be obtained, for example, by the process described in U.S. Pat. No. 4,911,903. The cores here are produced by hydrolytic polycondensation of tetraalkoxysilanes in an aqueous-ammoniacal medium, where firstly a sol of primary particles is produced, and the resultant SiO₂ particles are subsequently converted into the desired particle size by continuous, controlled metered addition of tetraalkoxysilane. This process enables the production of monodisperse SiO₂ cores having mean particle diameters of between 0.05 and 10 μm with a standard deviation of 5%.

Also preferred as starting material are SiO₂ cores which have been coated with (semi)metals or non-absorbent metal oxides, such as, for example, TiO₂, ZrO₂, ZnO₂, SnO₂ or Al₂O₃. The production of SiO₂ cores coated with metal oxides is described in greater detail in, for example, U.S. Pat. No. 5,846,310, DE 198 42 134 and DE 199 29 109.

The starting material employed can also be monodisperse cores of nonabsorbent metal oxides, such as TiO₂, ZrO₂, ZnO₂, SnO₂ or Al₂O₃, or metal-oxide mixtures. Their production is described, for example, in EP 0 644 914. Furthermore, the process of EP 0 216 278 for the production of monodisperse SiO₂ cores can readily be applied to other oxides with the same result. Tetraethoxysilane, tetrabutoxytitanium, tetrapropoxyzirconium or mixtures thereof are added in one portion, with vigorous mixing, to a mixture of alcohol, water and ammonia, whose temperature is set precisely to from 30 to 40° C. using a thermostat, and the resultant mixture is stirred vigorously for a further 20 seconds, giving a suspension of monodisperse cores in the nanometre region. After a postreaction time of from 1 to 2 hours, the cores are separated off in a conventional manner, for example by centrifugation, washed and dried.

Suitable starting materials for the production of the core/shell particles according to the invention are furthermore also monodisperse cores of polymers which contain included particles, for example metal oxides. Materials of this type are available, for example, from micro caps Entwicklungs- and Vertriebs GmbH in Rostock. Microencapsulations based on polyesters, polyamides and natural and modified carbohydrates are produced in accordance with customer-specific requirements.

It is furthermore possible to employ monodisperse cores of metal oxides which have been coated with organic materials, for example silanes. The monodisperse cores are dispersed in alcohols and modified with conventional organoalkoxysilanes. The silanisation of spherical oxide particles is also described in DE 43 16 814.

The cores of the core/shell particles according to the invention may, in addition, also comprise dyes, for example so-called nanocolorants, as described, for example, in WO 99/40123. The disclosure content of WO 99/40123 is hereby expressly included in the disclosure content of the present application.

For the intended use of the core/shell particles according to the invention for the preparation of diffractive colorants, it is important that the shell material can be formed into a film, i.e. that it can be softened, viscoelastically plasticised or liquefied by simple measures to such an extent that the cores of the core/shell particles are at least able to form domains having a regular arrangement. The regularly arranged cores in the matrix formed by film formation of the shells of the core/shell particles form a diffraction grating, which causes interference phenomena and thus results in very interesting colour effects.

The materials of core and shell may, as long as they satisfy the conditions indicated above, be of an inorganic, organic or even metallic character or they may be hybrid materials.

In view of the possibility of varying the invention-relevant properties of the cores of the core/shell particles according to the invention as needed, however, it is advantageous for the cores to comprise one or more polymers and/or copolymers (core polymers) or to consist of polymers of this type.

The cores preferably comprise a single polymer or copolymer. For the same reason, it is advantageous for the shells of the core/shell particles according to the invention likewise to comprise one or more polymers and/or copolymers (shell polymers; matrix polymers) or polymer precursors and, if desired, auxiliaries and additives, where the composition of the shells may be selected in such a way that they are essentially dimensionally stable and tack-free in a non-swelling environment at room temperature.

With the use of polymer substances as shell material and, if desired, core material, the person skilled in the art gains the freedom to determine their relevant properties, such as, for example, their composition, the particle size, the mechanical data, the refractive index, the glass transition temperature, the melting point and the core:shell weight ratio and thus also the applicational properties of the core/shell particles, which ultimately also affect the properties of the diffractive colorants prepared therefrom.

Polymers and/or copolymers which may be present in the core material or of which it consists are high-molecular-weight compounds which conform to the specification given above for the core material. Both polymers and copolymers of polymerisable unsaturated monomers and polycondensates and copolycondensates of monomers containing at least two reactive groups, such as, for example, high-molecular-weight aliphatic, aliphatic/aromatic or fully aromatic polyesters, polyamides, polycarbonates, polyureas and polyurethanes, but also amino and phenolic resins, such as, for example, melamine-formaldehyde, urea-formaldehyde and phenol-formaldehyde condensates, are suitable.

For the preparation of epoxy resins, which are likewise suitable as core material, epoxide prepolymers, which are obtained, for example, by reaction of bisphenol A or other bisphenols, resorcinol, hydroquinone, hexanediol or other aromatic or aliphatic diols or polyols, or phenol-formaldehyde condensates, or mixtures thereof with one another, with epichlorohydrin or other di- or polyepoxides, are usually mixed with further condensation-capable compounds directly or in solution and allowed to cure. The polymers of the core material are advantageously, in a preferred variant of the invention, crosslinked (co)polymers, since these usually only exhibit their glass transition at high temperatures. These crosslinked polymers may either already have been crosslinked during the polymerisation or polycondensation or copolymerisation or copolycondensation or may have been post-crosslinked in a separate process step after the actual (co)polymerisation or (co)polycondensation.

In principle, polymers of the classes already mentioned above, if they are selected or constructed in such a way that they conform to the specification given above for the shell polymers, are suitable for the shell material, like for the core material.

Polymers which meet the specifications for a shell material are likewise present in the groups of polymers and copolymers of polymerisable unsaturated monomers and polycondensates and copolycondensates of monomers containing at least two reactive groups, such as, for example, high-molecular-weight aliphatic, aliphatic/aromatic or fully aromatic polyesters and polyamides.

Taking into account the above conditions for the properties of the shell polymers (=matrix polymers), selected units from all groups of organic film formers are in principle suitable for their preparation. In particular if the matrix of the diffractive colorant is to be formed from crosslinked organic polymers, the shell polymers can be selected virtually as desired. It need only be ensured that these polymers can be provided with crosslinkable groups.

Some further examples are intended to illustrate the broad range of polymers which are suitable for the production of the shells.

If the shell is intended to have a comparatively low refractive index, polymers, such as polyethylene, polypropylene, polyethylene oxide, polyacrylates, polymethacrylates, polybutadiene, polymethyl methacrylate, polytetrafluoroethylene, polyoxymethylene, polyesters, polyamides, polyepoxides, polyurethane, rubber, polyacrylonitrile and polyisoprene, for example, are suitable.

If the shell is intended to have a comparatively high refractive index, polymers having a preferably aromatic basic structure, such as polystyrene, polystyrene copolymers, such as, for example, SAN, aromatic-aliphatic polyesters and polyamides, aromatic polysulfones and polyketones, polyvinyl chloride, polyvinylidene chloride and, on suitable selection of a high-refractive-index core material, also polyacrylonitrile or polyurethane, for example, are suitable for the shell.

In an embodiment of core/shell particles which is particularly preferred in accordance with the invention, the core consists of crosslinked polystyrene and the shell of a polyacrylate, preferably polyethyl acrylate and/or polymethyl methacrylate, to which crosslinkable monomers have been added.

With respect to the processability of the core/shell particles into diffractive colorants, it is advantageous for the core:shell weight ratio to be in the range from 2:1 to 1:5, preferably in the range from 3:2 to 1:3 and particularly preferably in the range from 1:1 to 2:3. In general, it is advantageous to increase the shell proportion if the particle diameter of the cores increases.

The core/shell particles to be employed in accordance with the invention can be produced by various processes. A preferred way of obtaining the particles is a process for the production of core/shell particles by a) surface treatment of monodisperse cores, and b) application of the shell of organic polymers to the treated cores.

In a process variant, the monodisperse cores are obtained in a step a1) by emulsion polymerisation.

In a preferred variant of the invention, a crosslinked polymeric interlayer, which preferably contains reactive centres to which the shell can be covalently bonded, is applied to the cores in step a), preferably by emulsion polymerisation or by ATR polymerisation. ATR polymerisation here stands for atom transfer radical polymerisation, as described, for example, in K. Mayjaszewski, Practical Atom Transfer Radical Polymerisation, Polym. Mater. Sci. Eng. 2001, 84. The encapsulation of inorganic materials by means of ATRP is described, for example, in T. Werne, T. E. Patten, Atom Transfer Radical Polymerisation from Nanoparticles: A Tool for the Preparation of Well-Defined Hybrid Nanostructures and for Understanding the Chemistry of Controlled/“Living” Radical Polymerisation from Surfaces, J. Am. Chem. Soc. 2001, 123, 7497-7505 and WO 00/11043. The performance both of this method and of emulsion polymerisations is familiar to the person skilled in the art of polymer preparation and is described, for example, in the above-mentioned literature references.

The liquid reaction medium in which the polymerisations or copolymerisations can be carried out consists of the solvents, dispersion media or diluents usually employed in polymerisations, in particular in emulsion polymerisation processes. The choice here is made in such a way that the emulsifiers employed for homogenisation of the core particles and shell precursors are able to develop adequate efficacy. Suitable liquid reaction media for carrying out the process according to the invention are aqueous media, in particular water.

Suitable for initiation of the polymerisation are, for example, polymerisation initiators which decompose either thermally or photochemically, form free radicals and thus initiate the polymerisation. Preferred thermally activatable polymerisation initiators here are those which decompose at between 20 and 180° C., in particular between 20 and 80° C. Particularly preferred polymerisation initiators are peroxides, such as dibenzoyl peroxide, di-tertbutyl peroxide, peresters, percarbonates, perketals, hydroperoxides, but also inorganic peroxides, such as H₂O₂, salts of peroxosulfuric acid and peroxodisulfuric acid, azo compounds, alkylboron compounds, and hydro-carbons which decompose homolytically. The initiators and/or photoinitiators, which, depending on the requirements of the polymerised material, are employed in amounts of between 0.01 and 15% by weight, based on the polymerisable components, can be used individually or, in order to utilise advantageous synergistic effects, in combination with one another. In addition, use is made of redox systems, such as, for example, salts of peroxodisulfuric acid and peroxosulfuric acid in combination with low-valency sulfur compounds, particularly ammonium peroxodisulfate in combination with sodium dithionite.

Corresponding processes have also been described for the production of polycondensation products. Thus, it is possible for the starting materials for the preparation of polycondensation products to be dispersed in inert liquids and condensed, preferably with removal of low-molecular-weight reaction products, such as water or—for example on use of di(lower alkyl) dicarboxylates for the preparation of polyesters or polyamides—lower alkanols.

Polyaddition products are obtained analogously by reaction of compounds which contain at least two, preferably three, reactive groups, such as, for example, epoxide, cyanate, isocyanate or isothiocyanate groups, with compounds carrying complementary reactive groups. Thus, isocyanates react, for example, with alcohols to give urethanes, with amines to give urea derivatives, while epoxides react with these complementary groups to give hydroxyethers and hydroxylamines respectively. Like the polycondensations, polyaddition reactions can also advantageously be carried out in an inert solvent or dispersion medium.

It is also possible for aromatic, aliphatic or mixed aromatic/aliphatic polymers, for example polyesters, polyurethanes, polyamides, polyureas, polyepoxides or also solution polymers, to be dispersed or emulsified (secondary dispersion) in a dispersion medium, such as, for example, in water, alcohols, tetrahydrofuran or hydrocarbons, and to be post-condensed, crosslinked and cured in this fine distribution.

The stable dispersions required for these polymerisation, polycondensation or polyaddition processes are generally prepared using dispersion auxiliaries.

The dispersion auxiliaries used are preferably water-soluble, high-molecular-weight organic compounds containing polar groups, such as polyvinylpyrrolidone, copolymers of vinyl propionate or acetate and vinylpyrrolidone, partially saponified copolymers of an acrylate and acrylonitrile, polyvinyl alcohols having different residual acetate contents, cellulose ethers, gelatine, block copolymers, modified starch, low-molecular-weight polymers containing carboxyl and/or sulfonyl groups, or mixtures of these substances.

Particularly preferred protective colloids are polyvinyl alcohols having a residual acetate content of less than 35 mol %, in particular from 5 to 39 mol %, and/or vinylpyrrolidone-vinyl propionate copolymers having a vinyl ester content of less than 35% by weight, in particular from 5 to 30% by weight.

It is possible to use nonionic or ionic emulsifiers, if desired also as a mixture. Preferred emulsifiers are optionally ethoxylated or propoxylated, relatively long-chain alkanols or alkylphenols having different degrees of ethoxylation or propoxylation (for example adducts with from 0 to 50 mol of alkylene oxide) or neutralised, sulfated, sulfonated or phosphated derivatives thereof. Neutralised dialkylsulfosuccinic acid esters or alkyldiphenyl oxide disulfonates are also particularly suitable.

Particularly advantageous are combinations of these emulsifiers with the above-mentioned protective colloids, since particularly finely divided dispersions are obtained therewith.

Special processes for the production of monodisperse polymer particles have also already been described in the literature (for example R. C. Backus, R. C. Williams, J. Appl. Physics 19, p. 1186 (1948)) and can advantageously be employed, in particular, for the production of the cores. It need merely be ensured here that the above-mentioned particle sizes are observed. A further aim is the greatest possible uniformity of the polymers. The particle size in particular can be set via the choice of suitable emulsifiers and/or protective colloids or corresponding amounts of these compounds.

Through the setting of the reaction conditions, such as temperature, pressure, reaction duration and use of suitable catalyst systems, which influence the degree of polymerisation in a known manner, and the choice of the monomers employed for their preparation—in terms of type and proportion—the desired property combinations of the requisite polymers can be set specifically.

Monomers which result in polymers having a high refractive index are generally those which contain aromatic moieties or those which contain heteroatoms having a high atomic number, such as, for example, halogen atoms, in particular bromine or iodine atoms, sulfur or metal ions, i.e. atoms or atomic groups which increase the polarisability of the polymers.

Polymers having a low refractive index are accordingly obtained from monomers or monomer mixtures which do not contain the said moieties and/or atoms of high atomic number or only do so in a small proportion.

A review of the refractive indices of various common homopolymers is given, for example, in Ullmanns Encyklopädie der technischen Chemie [Ullmann's Encyclopaedia of Industrial Chemistry], 5th Edition, Volume A21, page 169. Examples of monomers which can be polymerised by means of free radicals and result in polymers having a high refractive index are:

Group a): styrene, styrenes which are alkyl-substituted on the phenyl ring, α-methylstyrene, mono- and dichlorostyrene, vinylnaphthalene, isopropenylnaphthalene, isopropenylbiphenyl, vinylpyridine, isopropenylpyridine, vinylcarbazole, vinylanthracene, N-benzylmethacrylamide and p-hydroxymethacrylanilide.

Group b): acrylates containing aromatic side chains, such as, for example, phenyl (meth)acrylate (=abbreviated notation for the two compounds phenyl acrylate and phenyl methacrylate), phenyl vinyl ether, benzyl (meth)acrylate, benzyl vinyl ether, and compounds of the formulae:

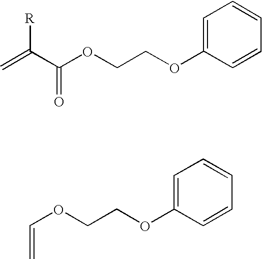

In order to improve clarity and simplify the notation of carbon chains in the formulae above and below, only the bonds between the carbon atoms are shown. This notation corresponds to the depiction of aromatic cyclic compounds, where, for example, benzene is depicted by a hexagon with alternating single and double bonds.

Also suitable are compounds containing sulfur bridges instead of oxygen bridges, such as, for example:

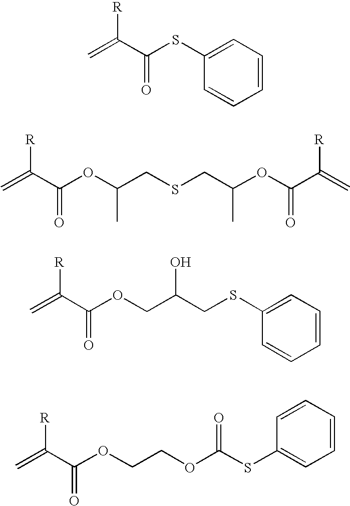

In the above formulae, R is hydrogen or methyl. The phenyl rings in these monomers may carry further substituents. Such substituents are suitable for modifying the properties of the polymers produced from these monomers within certain limits. They can therefore be used in a targeted manner to optimise, in particular, the applicationally relevant properties of the diffractive colorants according to the invention.

Suitable substituents are, in particular, halogen, NO₂, alkyl having from one to twenty carbon atoms, preferably methyl, alkoxy having from one to twenty carbon atoms, carboxyalkyl having from one to twenty carbon atoms, carbonylalkyl having from one to twenty carbon atoms or —OCOO-alkyl having from one to twenty carbon atoms. The alkyl chains in these radicals may themselves optionally be substituted or interrupted by divalent heteroatoms or groups, such as, for example, —O—, —S—, —NH—, —COO—, —OCO— or —OCOO—, in non-adjacent positions.

Group c): monomers containing heteroatoms, such as, for example, vinyl chloride, acrylonitrile, methacrylonitrile, acrylic acid, methacrylic acid, acrylamide and methacrylamide, or organometallic compounds, such as, for example:

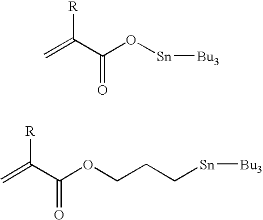

Group d): an increase in the refractive index of the polymers is also achieved by copolymerisation of carboxyl-containing monomers and conversion of the resultant “acidic” polymers into the corresponding salts with metals of relatively high atomic weight, such as, for example, preferably with K, Ca, Sr, Ba, Zn, Pb, Fe, Ni, Co, Cr, Cu, Mn, Sn or Cd.

The above-mentioned monomers, which make a considerable contribution towards the refractive index of the polymers produced therefrom, can be homopolymerised or copolymerised with one another. They can also be copolymerised with a certain proportion of monomers which make a lesser contribution towards the refractive index. Such copolymerisable monomers having a lower refractive index contribution are, for example, acrylates, methacrylates, or vinyl ethers or vinyl esters containing purely aliphatic radicals.

In addition, crosslinking agents which can be employed for the production of crosslinked polymer cores from polymers produced by means of free radicals or for the subsequent crosslinking of the matrix of shell material are also all bifunctional or polyfunctional compounds which are copolymerisable with the above-mentioned monomers or which can subsequently react with the polymers with crosslinking.

Examples of suitable crosslinking agents are presented below, divided into groups for systematisation:

Group 1: bisacrylates, bismethacrylates and bisvinyl ethers of aromatic or aliphatic di- or polyhydroxyl compounds, in particular of butanediol (butanediol di(meth)acrylate, butanediol bisvinyl ether), hexanediol (hexanediol di(meth)acrylate, hexanediol bisvinyl ether), pentaerythritol, hydroquinone, bishydroxyphenylmethane, bishydroxyphenyl ether, bishydroxymethylbenzene, bisphenol A or with ethylene oxide spacers, propylene oxide spacers or mixed ethylene oxide/propylene oxide spacers.

Further crosslinking agents from this group are, for example, di- or polyvinyl compounds, such as divinylbenzene, or methylenebisacrylamide, triallyl cyanurate, divinylethyleneurea, trimethylolpropane tri(meth)acrylate, trimethylolpropane trivinyl ether, pentaerythritol tetra(meth)acrylate, pentaerythritol tetravinyl ether, and crosslinking agents having two or more different reactive ends, such as, for example, (meth)allyl (meth)acrylates of the formulae: 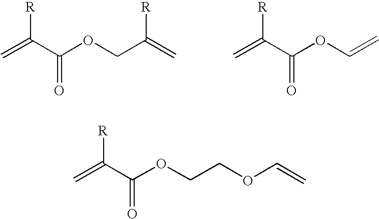
in which R is hydrogen or methyl.

Group 2: reactive crosslinking agents which act in a crosslinking manner, but in most cases in a post-crosslinking manner, for example during warming or drying, and which are copolymerised into the core or shell polymers as copolymers.

Examples thereof are: N-methylol(meth)acrylamide, acrylamidoglycolic acid, and ethers and/or esters thereof with C₁- to C₆-alcohols, diacetoneacrylamide (DAAM), glycidyl methacrylate (GMA), methacryloyloxypropyltrimethoxysilane (MEMO), vinyltrimethoxysilane and m-isopropenylbenzyl isocyanate (TMI).

Group 3: carboxyl groups which have been incorporated into the polymer by copolymerisation of unsaturated carboxylic acids are crosslinked in a bridge-like manner via polyvalent metal ions. The unsaturated carboxylic acids employed for this purpose are preferably acrylic acid, methacrylic acid, maleic anhydride, itaconic acid and fumaric acid. Suitable metal ions are Mg, Ca, Sr, Ba, Zn, Pb, Fe, Ni, Co, Cr, Cu, Mn, Sn and Cd. Particular preference is given to Ca, Mg and Zn, Ti and Zr.

Group 4: post-crosslinked additives, which are taken to mean bis- or polyfunctionalised additives which react irreversibly with the polymer (by addition or preferably condensation reactions) with formation of a network. Examples thereof are compounds which contain at least two of the following reactive groups per molecule: epoxide, aziridine, isocyanate, acid chloride, carbodiimide or carbonyl groups, furthermore, for example, 3,4-dihydroxyimidazolinone and derivatives thereof (Fixapret products from BASF).

As already explained above, post-crosslinking agents containing reactive groups, such as, for example, epoxide and isocyanate groups, require complementary reactive groups in the polymer to be crosslinked. Thus, isocyanates react, for example, with alcohols to give urethanes, with amines to give urea derivatives, while epoxides react with these complementary groups to give hydroxyethers and hydroxyamines respectively.

The term post-crosslinking is also taken to mean photochemical curing or oxidative or air- or moisture-induced curing of the systems.

The above-mentioned monomers and crosslinking agents can be combined and (co)polymerised with one another as desired and in a targeted manner in such a way that an optionally crosslinked (co)polymer having the desired refractive index and the requisite stability criteria and mechanical properties is obtained.

It is also possible additionally to copolymerise further common monomers, for example acrylates, methacrylates, vinyl esters, butadiene, ethylene or styrene, in order, for example, to set the glass transition temperature or the mechanical properties of the core and/or shell polymers as needed.

It is likewise preferred in accordance with the invention for the application of the shell of organic polymers to be carried out by grafting, preferably by emulsion polymerisation or ATR polymerisation. The methods and monomers described above can be employed correspondingly here.

In particular on use of inorganic cores, it may also be preferred for the core to be subjected to a pre-treatment which enables binding of the shell before the shell is polymerised on. This can usually consist in chemical functionalisation of the particle surface, as is known from the literature for a very wide variety of inorganic materials. It may particularly preferably involve application to the surface of chemical functions which, as active chain end, enable grafting-on of the shell polymers. Examples which may be mentioned in particular here are terminal double bonds, epoxy functions and polycondensable groups. The functionalisation of hydroxyl-carrying surfaces with polymers is disclosed, for example, in EP-A-337 144. Further methods for the modification of particle surfaces are well known to the person skilled in the art and are described, for example, in various textbooks, such as Unger, K. K., Porous Silica, Elsevier Scientific Publishing Company (1979).

In a likewise preferred embodiment of the present invention, the diffractive colorants comprise at least one contrast material. The contrast materials effect an increase in brightness, contrast and depth of the observed colour effects in the diffractive colorants according to the invention. For the purposes of the invention, the term contrast materials is taken to mean all materials which cause a strengthening of this type in the optical effect. These contrast materials are usually pigments.

For the purposes of the present invention, the term pigments here is taken to mean any solid substance which exhibits an optical effect in the visible wavelength region of light. In accordance with the invention, the term pigments is applied here, in particular, to substances which conform to the definition of pigments in accordance with DIN 55943 or DIN 55945. According to this definition, a pigment is an inorganic or organic, coloured or non-coloured colorant which is virtually insoluble in the application medium. Both inorganic and organic pigments can be employed in accordance with the invention.

Pigments can be divided into absorption pigments and lustre pigments in accordance with their physical mode of functioning. Absorption pigments are pigments which absorb at least part of visible light and therefore cause a colour impression and in the extreme case appear black. According to DIN 55943 or DIN 55944, lustre pigments are pigments in which lustre effects arise through directed reflection at metallic or strongly light-refracting pigment particles which are formed and aligned in a predominantly two-dimensional manner. These standards define interference pigments as lustre pigments whose colouring action is based entirely or predominantly on the phenomenon of interference. In particular, these are so-called mother-of-pearl pigments or fire-coloured metal bronzes. Of economic importance amongst the interference pigments are also, in particular, the pearlescent pigments, which consist of colourless, transparent and highly light-refracting platelets. Depending on the orientation in a matrix, they produce a soft lustre effect which is known as pearlescence. Examples of pearlescent pigments are guanine-containing pearl essence, pigments based on lead carbonates, bismuth oxide chloride or titanium dioxide mica. In particular, the titanium dioxide micas, which are distinguished by mechanical, chemical and thermal stability, are frequently employed for decorative purposes.

In accordance with the invention, it is possible to employ both absorption and lustre pigments, it also being possible, in particular, to employ interference pigments. It has been found that the use of absorption pigments is preferred, in particular for increasing the intensity of the optical effects. Both white and coloured or black pigments can be employed here, where the term coloured pigments is taken to mean all pigments which give a colour impression other than white or black, such as, for example, Heliogen™ Blue K 6850 (BASF, Cu phthalocyanine pigment), Heliogen™ Green K 8730 (BASF, Cu phthalocyanine pigment), Bayferrox™ 105 M (Bayer, iron oxide-based red pigment) or Chromium Oxide Green GN-M (Bayer, chromium oxide-based green pigment). Owing to the colour effects achieved, preference is in turn given amongst the absorption pigments to the black pigments. For example, mention may be made here of pigment carbon black (for example the carbon black product line from Degussa (in particular Purex™ LS 35 and Corax™ N 115)) and iron oxide black, manganese black as well as cobalt black and antimony black. Black mica grades can also advantageously be employed as black pigment (for example Iriodin™ 600, Merck; iron oxide-coated mica).

It has been found that it is advantageous in accordance with the invention if the particle size of the at least one contrast material is at least twice as large as the particle size of the core material. If the particles of the contrast material are smaller, only inadequate optical effects are achieved. It is assumed that smaller particles interfere with the arrangement of the cores in the matrix and cause a change in the lattice which forms. The particles preferably employed in accordance with the invention, which have a size which is at least twice that of the cores, only interact locally with the lattice formed from the cores. Electron photomicrographs confirm that the incorporated particles only interfere with the lattice of core particles to a small extent, or not at all. The term particle size of the contrast materials, which are frequently also flake-form as pigments, is in each case taken to mean here the largest dimension of the particles. If flake-form pigments have a thickness in the region of the particle size of the cores or even below, the present studies show that this does not interfere with the lattice orders. It has also been found that the shape of the incorporated contrast material particles has little or no influence on the optical effect. Both spherical and flake-form and needle-shaped contrast materials can be incorporated in accordance with the invention. The only factor of significance appears to be the absolute particle size in relation to the particle size of the cores. It is therefore preferred for the purposes of the invention if the particle size of the at least one contrast material is at least twice as large as the particle size of the core material, where the particle size of the at least one contrast material is preferably at least four times as large as the particle size of the core material, since the observable interactions are then even smaller.

A sensible upper limit for the particle size of the contrast materials arises from the limit at which the individual particles themselves become visible or impair the mechanical properties of the diffractive colorant owing to their particle size. Determination of this upper limit presents no difficulties at all to the person skilled in the art.

Also of importance for the effect desired in accordance with the invention is the amount of contrast material employed. It has been found that effects are usually observed if at least 0.05% by weight of contrast material, based on the weight of the diffractive colorant, is employed. It is particularly preferred for the diffractive colorant to comprise at least 0.2% by weight and especially preferably at least 1% by weight of contrast material since these increased contents of contrast material generally also result, in accordance with the invention, in more intense effects.

Conversely, relatively large amounts of contrast material under certain circumstances adversely affect the processing properties of the core/shell particles and thus make the preparation of diffractive colorants according to the invention more difficult. In addition, it is expected that the formation of the lattice of core particles will be interfered with above a certain proportion of contrast material, which is dependent on the particular material, and instead oriented contrast material layers will form. It is therefore preferred in accordance with the invention for the diffractive colorant to comprise a maximum of 20% by weight of contrast material, based on the weight of the diffractive colorant, it being particularly preferred for the diffractive colorant to comprise a maximum of 12% by weight and especially preferably a maximum of 5% by weight of contrast material.

In a particular embodiment of the present invention, however, it may also be preferred for the diffractive colorants to comprise the largest possible amounts of contrast material. This is the case, in particular, if the contrast material is at the same time intended to increase the mechanical strength of the diffractive colorant.

The diffractive colorants according to the invention are preferably flake-form particles whose thickness is preferably in the range from 0.5 to 20 μm, particularly preferably in the range from 1 to 10 μm, where at least 5, preferably at least 8 and particularly preferably at least 10 core layers follow one another in each spatial direction in the flake.

The diffractive colorants according to the invention can be obtained by grinding or crushing films of core/shell particles whose production is described in principle in DE-A-10145450. Consequently, a suitable process for the preparation of diffractive colorants is one in which a film having a brittle matrix is produced from core/shell particles whose core is essentially solid and has an essentially monodisperse size distribution, where a difference exists between the refractive indices of the core material and of the shell material, and the film is subsequently comminuted to give particles, preferably ground.

In a preferred process, use is made of core/shell particles whose shell consists of polymers which have a glass transition temperature T_{G} of above 25° C., preferably above 50° C. and particularly preferably above 70° C. Corresponding polymers are well known to the person skilled in the art and are given, for example, in Brandrup, J. (Ed.).: Polymer Handbook. Chichester Wiley 1966. Preferred shell materials here are homopolymeric or copolymeric poly(cyclohexyl methacrylate), polystyrene and substituted polystyrene derivatives, such as, for example, poly(iodostyrene) and poly(bromostyrene), polyacrylates and polymethacrylates having a Tg above the use temperature, polyvinyl chloride of high Tg and other vinylic polymers which arise through conversion of polyvinyl acetate, and polyacrylonitrile and styrene-acrylonitrile copolymers.

In another preferred process, use is made of core/shell particles whose shell consists of polymers which have a crosslinkable functionality. In this process variant, post-crosslinking additives are preferably added to the core/shell particles before production of the film, and the actual crosslinking is carried out after production of the film. The post-crosslinking agent or crosslinkable functionality employed here can be, in particular, the compounds or monomers described above. The crosslinking here can be initiated by the methods described, with thermal activation being particularly preferred for the post-crosslinking.

In the production of the films, a mixture of core/shell particles and optionally further additives is firstly prepared. The mixture is preferably then subjected to a mechanical force at a temperature at which the shell is flowable.

In a preferred variant of the production of films according to the invention, the temperature at which the mixture is subjected to the mechanical force is at least 40° C., preferably at least 60° C., above the glass transition temperature of the shell of the core/shell particles. It has been found empirically that the flowability of the shell in this temperature range meets the requirements for economical production of the films to a particular extent.

In a likewise preferred process variant which results in the films according to the invention, the flowable mixtures are cooled under the action of the mechanical force to a temperature at which the shell is no longer flowable.

For the purposes of the present invention, the action of mechanical force can be the action of a force which occurs in the conventional processing steps of polymers. In preferred variants of the present invention, the action of mechanical force takes place either:
through uniaxial pressing or
during a transfer moulding operation,
during (co)extrusion or
during a calendering operation or
during a blowing operation.

If the action of force takes place through uniaxial pressing, the films according to the invention are preferably films. Films according to the invention can preferably also be produced by calendering, film blowing or flat-film extrusion. The various ways of processing polymers under the action of mechanical forces are well known to the person skilled in the art and are revealed, for example, by the standard textbook Adolf Franck, “KunststoffKompendium” [Plastics Compendium]; Vogel-Verlag; 1996.

The films according to the invention may, if it is technically advantageous, comprise auxiliaries and additives here. They can serve for optimum setting of the applicational data or properties desired or necessary for application and processing. Examples of auxiliaries and/or additives of this type are plasticisers, film-formation auxiliaries, flow-control agents, fillers, melting assistants, adhesives, release agents, application auxiliaries and viscosity modifiers, for example thickeners.

Particularly recommended are additions of film-formation auxiliaries and film modifiers based on compounds of the general formula HO—CₙH₂ₙO—(CₙH₂ₙ—O)ₘH, in which n is a number from 2 to 4, preferably 2 or 3, and m is a number from 0 to 500. The number n can vary within the chain, and the various chain members can be incorporated in a random or blockwise distribution. Examples of auxiliaries of this type are ethylene glycol, propylene glycol, di-, tri- and tetraethylene glycol, di-, tri- and tetrapropylene glycol, polyethylene oxides, polypropylene oxide and ethylene oxide-propylene oxide copolymers having molecular weights of up to about 15.000 and a random or block-like distribution of the ethylene oxide and propylene oxide units.

If desired, organic or inorganic solvents, dispersion media or diluents, which, for example, extend the open time of the formulation, i.e. the time available for its application to substrates, waxes or hot-melt adhesives are also possible as additives.

If desired, UV and weathering stabilisers can also be added to the films. Suitable for this purpose are, for example, derivatives of 2,4-dihydroxybenzophenone, derivatives of 2-cyano-3,3′-diphenyl acrylate, derivatives of 2,2′,4,4′-tetrahydroxybenzophenone, derivatives of o-hydroxyphenylbenzotriazole, salicylic acid esters, o-hydroxyphenyl-s-triazines or sterically hindered amines. These substances may likewise be employed individually or in the form of a mixture.

The total amount of auxiliaries and/or additives is up to 40% by weight, preferably up to 20% by weight, particularly preferably up to 5% by weight, of the weight of the films. Accordingly, the films consist of at least 60% by weight, preferably at least 80% by weight and particularly preferably at least 95% by weight, of core/shell particles.

These films can be employed as diffractive colorants according to the invention. If the aim is to prepare particulate diffractive colorants, this is successfully carried out by comminuting the films by chopping, crushing and/or grinding to give pigments of suitable size. This operation can be carried out, for example, in a continuous belt process. A roll mill or grinding mill can be employed. If particularly uniform particle sizes are desired for the diffractive colorants, the comminution step may be followed by one or more sieving steps.

The inverse structure to the opal structure is notionally formed by regular spherical cavities being arranged in closest sphere packing in a solid material. An advantage of inverse structures of this type over the normal structures is the formation of photonic band gaps with much lower dielectric constant contrasts still (K. Busch et al. Phys. Rev. Letters E, 198, 50, 3896).

Consequently, diffractive colorants with cavities must have a solid matrix. In a variant of the invention, the matrix essentially consists of an organic polymer, which is preferably crosslinked. In another variant of the invention, the matrix around the cavities essentially consists of an inorganic material, preferably a metal or semimetal or a metal chalcogenide or metal pnictide, particular preference being given to silicon dioxide, aluminium oxide, zirconium oxide, iron oxides, titanium dioxide, cerium dioxide, gallium nitride, boron nitride, aluminium nitride, silicon nitride and phosphorus nitride, or mixtures thereof.

Three-dimensional inverse structures, i.e. diffractive colorants having regular arrangements of cavities which are to be employed in accordance with the invention, can be produced, for example, by template synthesis:
Monodisperse spheres are arranged in closest sphere packing as structure-forming templates.
The cavities between the spheres are filled with a gaseous or liquid precursor or a solution of a precursor utilising capillary effects.
The precursor is converted (thermally) into the desired material.
The templates are removed, leaving behind the inverse structure.

Many such processes are disclosed in the literature. For example, SiO₂ spheres can be arranged in closest sphere packing and the cavities filled with tetraethyl orthotitanate-containing solutions. After a number of conditioning steps, the spheres are removed using HF in an etching process, leaving behind the inverse structure of titanium dioxide (V. Colvin et al. Adv. Mater. 2001, 13, 180).

De La Rue et al. (De La Rue et al. Synth. Metals, 2001, 116, 469) describe the production of inverse opals consisting of TiO₂ by the following method: a dispersion of 400 nm polystyrene spheres is dried on a filter paper under an IR lamp. The filter cake is washed by sucking through ethanol, transferred into a glove box and infiltrated with tetraethyl orthotitanate by means of a water-jet pump. The filter paper is carefully removed from the latex/ethoxide composite, and the composite is transferred into a tubular furnace. Calcination in a stream of air is carried out in the tubular furnace at 575° C. for 8 hours, causing the formation of titanium dioxide from the ethoxide and burning out the latex particles. An inverse opal structure of TiO₂ remains behind.

Martinelli et al. (M. Martinelli et al. Optical Mater. 2001, 17, 11) describe the production of inverse TiO₂ opals using 780 nm and 3190 nm polystyrene spheres. A regular arrangement in spherical closest sphere packing is achieved by centrifuging the aqueous sphere dispersion at 700-1000 rpm for 24-48 hours followed by decantation and drying in air. The regularly arranged spheres are moistened with ethanol on a filter in a Büchner funnel and then provided dropwise with an ethanolic solution of tetraethyl orthotitanate. After the titanate solution has percolated in, the sample is dried in a vacuum desiccator for 4-12 hours. This filling procedure is repeated 4 to 5 times. The polystyrene spheres are subsequently burnt out at 600° C.-800° C. for 8-10 hours.

Stein et al. (A. Stein et al. Science, 1998, 281, 538) describe the synthesis of inverse TiO₂ opals starting from polystyrene spheres having a diameter of 470 nm as templates. These are produced in a 28-hour process, subjected to centrifugation and air-dried. The latex templates are then applied to a filter paper. Ethanol is sucked into the latex template via a Buchner funnel connected to a vacuum pump. Tetraethyl orthotitanate is then added dropwise with suction. After drying in a vacuum desiccator for 24 hours, the latices are burnt out at 575° C. for 12 hours in a stream of air.

Vos et al. (W. L. Vos et al. Science, 1998, 281, 802) produce inverse TiO₂ opals using polystyrene spheres having diameters of 180-1460 nm as templates. In order to establish closest packing of the spheres, a sedimentation technique is used supported by centrifugation over a period of up to 48 hours. After slow evacuation in order to dry the template structure, an ethanolic solution of tetra-n-propoxy orthotitanate is added to the latter in a glove box. After about 1 hour, the infiltrated material is brought into the air in order to allow the precursor to react to give TiO₂. This procedure is repeated eight times in order to ensure complete filling with TiO₂. The material is then calcined at 450° C.

Core/shell particles whose shell forms a matrix and whose core is essentially solid and has an essentially monodisperse size distribution are described in German patent application DE-A-10145450. The use of core/shell particles whose shell forms a matrix and whose core is essentially solid and has an essentially monodisperse size distribution as templates for the production of inverse opal structures and a process for the production of inverse opal-like structures using core/shell particles of this type are described in international patent application WO 2004/031102. The mouldings described having homogeneous, regularly arranged cavities (i.e. inverse opal structure) preferably have walls of metal oxides or of elastomers. Consequently, the mouldings described are either hard and brittle or exhibit an elastomeric character.

The earlier German patent application with the applicational file reference DE 10341198.4 describes the use of core/shell particles whose shell has thermoplastic properties for the production of mouldings having homogeneous, regularly arranged cavities whose mechanical properties are particularly advantageous.

Core/shell particles whose shell forms a matrix and whose core is essentially solid and has an essentially monodisperse size distribution and whose shell is bonded to the core via an interlayer and whose shell has thermoplastic properties are used here for the production of mouldings having homogeneous, regularly arranged cavities. The corresponding process for the production of mouldings having homogeneous, regularly arranged cavities is characterised in that core/shell particles whose shell forms a matrix and whose core is essentially solid and has an essentially monodisperse size distribution and is bonded to the core via an interlayer and whose shell has thermoplastic properties are converted into mouldings, preferably films, with application of a mechanical force and elevated temperature, and the cores are subsequently removed.

This gives mouldings having homogeneous, regularly arranged cavities which are characterised in that the regularly arranged cavities are embedded in a matrix having thermoplastic or thermosetting properties.

Owing to the relative ease of processing, it is particularly preferred in a variant for the regularly arranged cavities to be embedded in a matrix having thermoplastic properties, where the matrix is preferably built up from poly(styrene), thermoplastic poly(acrylate) derivatives, preferably poly(methyl methacrylate) or poly(cyclohexyl methacrylate), or thermoplastic copolymers of these polymers with other acrylates, such as, preferably, styrene-acrylonitrile copolymers, styrene-ethyl acrylate copolymers or methyl methacrylate-ethyl acrylate copolymers.

In another preferred embodiment, the core in the core/shell particles essentially consists of a UV radiation-degradable material, preferably a UV-degradable organic polymer and particularly preferably poly(tert-butyl methacrylate), poly(methyl methacrylate), poly(n-butyl methacrylate) or copolymers containing one of these polymers.

The shell of the core/shell particles preferably consists here of the material that later forms the matrix around the cavities.

The cores can be removed by various methods. If the cores consist of suitable inorganic materials, they can be removed by etching. For example, silicon dioxide cores can preferably be removed using HF, in particular dilute HF solution. In this procedure, it may in turn be preferred for crosslinking of the shell to be carried out before or after removal of the cores. In this case, the shell and thus the matrix of the moulding attains thermosetting properties. If the cores in the core/shell particles are built up from a UV-degradable material, preferably a UV-degradable organic polymer, the cores are removed by UV irradiation. In this procedure too, it may in turn be preferred for crosslinking of the shell to be carried out before or after removal of the cores.

The use of core/shell particles whose shell forms a matrix and whose core essentially consists of a degradable polymer and has an essentially monodisperse size distribution and whose shell can be pyrolysed to give a carbon matrix for the production of mouldings having regularly arranged cavities, a process for the production of mouldings having regularly arranged cavities and the corresponding mouldings are described in the earlier German patent application DE 10357681.9.

The term carbon matrix here is taken to mean materials which substantially correspond to those of carbon fibres. In an extreme case, the carbon matrix according to the invention is elemental carbon, preferably in amorphous or partially crystalline form, where the crystalline fractions are in the graphite modification or graphite-like modifications, such as fullerenes, carbon nanotubes and similar graphite-like structures. In another extreme variant of the present invention, the carbon matrix comprises conductor polymers, such as, for example, polyimides, which form on thermal condensation of polyacrylonitrile. In general, however, the carbon matrix is a material whose chemical structure lies between these two extremes. It is assumed that, in a similar manner to the situation in carbon blacks, varying proportions of polycyclic aromatic hydrocarbons provided with imidic functions can be present in the materials.

In order to simplify the formation of the carbon matrix, it is particularly preferred in accordance with the invention for the shell in the core/shell particles to consist of essentially uncrosslinked organic polymers which are grafted onto the core via an at least partially crosslinked interlayer, where the shell is preferably formed essentially from polyacrylonitrile (PAN) or polymethacrylonitrile or copolymers containing polyacrylonitrile or polymethacrylonitrile, such as polystyrene-acrylonitrile (PSAN). PAN decomposes here at temperatures as low as 250-280° C. to form a suitable carbon matrix.

The process for the production of mouldings having regularly arranged cavities in accordance with DE 10357681.9 is characterised in that core/shell particles whose shell forms a matrix and whose core essentially consists of a degradable polymer and has an essentially monodisperse size distribution and whose shell can be pyrolysed to give a carbon matrix are converted into mouldings, preferably films, with application of a mechanical force and elevated temperature, and the cores are subsequently removed by degradation and the shell is decomposed at elevated temperature to give a carbon matrix.

It is particularly preferred for the degradable core in the core/shell particles to be thermally degradable and to consist of polymers which are either thermally depolymerisable, i.e. decompose into their monomers on exposure to heat, or for the core to consist of polymers which decompose on degradation to give low-molecular-weight constituents which are different from the monomers. It is important here that the degradation of the core polymers takes place at a temperature which is equal to or lower than the temperature at which the carbon matrix forms. Suitable polymers are given, for example, in the table “Thermal Degradation of Polymers” in Brandrup, J. (Ed.).: Polymer Handbook. Chichester Wiley 1966, pp. V-6-V-10, all polymers which give volatile degradation products being suitable. The contents of this table are expressly part of the disclosure content of the present application.

Suitable thermally degradable polymers are, in particular,
poly(styrene) and derivatives, such as poly(α-methylstyrene) or poly(styrene) derivatives carrying substituents on the aromatic ring, such as, in particular, partially or perfluorinated derivatives,
poly(acrylate) and poly(methacrylate) derivatives as well as esters thereof, particularly preferably poly(methyl methacrylate) or poly(cyclohexyl methacrylate), or copolymers of these polymers with other degradable polymers, such as, preferably, styrene-ethyl acrylate copolymers or methyl methacrylate-ethyl acrylate copolymers,
polybutadiene and copolymers with other monomers mentioned here,
cellulose and derivatives, such as oxidated cellulose and cellulose triacetate,
polyketones, such as, for example, poly(methyl isopropenyl ketone) or poly(methyl vinyl ketone),
polyolefins, such as, for example, polyethylene and polypropylene, polyisoprene, polyolefin oxides, such as, for example, polyethylene oxide or polypropylene oxide, polyethylene terephthalate, polyformaldehyde, polyamides, such as nylon 6 and nylon 66, polyperfluoroglucarodiamidine, polyperfluoropolyolefins, such as polyperfluoropropylene and polyperfluoroheptene,
polyvinyl acetate, polyvinyl chloride, polyvinyl alcohol, polyvinylcyclohexanone, polyvinyl butyrate and polyvinyl fluoride.

Particular preference is given here to the use of poly(styrene) and derivatives, such as poly(α-methylstyrene) or poly(styrene) derivatives carrying substituents on the aromatic ring, such as, in particular, partially or perfluorinated derivatives, poly(acrylate) and poly(methacrylate) derivatives as well as esters thereof, particularly preferably poly(methyl methacrylate) or poly(cyclohexyl methacrylate), or copolymers of these polymers with other degradable polymers, such as, preferably, styrene-ethyl acrylate copolymers or methyl methacrylate-ethyl acrylate copolymers, and polyolefins, polyolefin oxides, polyethylene terephthalate, polyformaldehyde, polyamides, polyvinyl acetate, polyvinyl chloride or polyvinyl alcohol.

If the core of the core/shell particles is to be degraded by UV radiation, the core is essentially built up from a UV radiation-degradable material, preferably a UV-degradable organic polymer and particularly preferably poly(tert-butyl methacrylate), poly(methyl methacrylate), poly(n-butyl methacrylate) or copolymers containing one of these polymers.

The present application now relates in one aspect to the use of the mouldings resulting from the production in accordance with the teaching of WO 2004/031102, DE 10357681.9 or DE 10341198.4 in cosmetics. Regarding the description of the mouldings and the process for the production of mouldings, reference is made to the patent applications WO 2004/031102 and DE 10341198.4, the corresponding disclosure content of which is expressly also part of the content of the present application.

It is particularly preferred in accordance with the invention for the average diameter of the cavities in the mouldings to be in the range of about 50-500 nm, preferably in the range of 100-500 nm and very particularly preferably in the range from 200 to 280 nm.

The mouldings are either produced directly in powder form in the corresponding processes or can be comminuted by grinding. The resultant particles are then used in the sense according to the invention as diffractive colorants for decorative use in cosmetic compositions or on the skin.

The concentration of the diffractive colorant in the application system to be pigmented, i.e. the dispersion, is generally between 0.1 and 70% by weight, preferably between 0.1 and 50% by weight and in particular between 1.0 and 20% by weight, based on the total solids content of the system.

The present invention furthermore relates to compositions for topical application comprising at least one diffractive colorant and at least one carrier which is suitable for topical application. Preference is given here to compositions in which the total amount of the diffractive colorant(s) is selected from the range from 0.1% by weight to 30% by weight, based on the total weight of the composition.

Furthermore, the compositions according to the invention may also comprise further dyes and coloured pigments. The dyes and coloured pigments can be selected from the corresponding positive list in the German Cosmetics Regulation or the EU list of cosmetic colorants. In most cases, they are identical with the dyes approved for foods. Advantageous coloured pigments are, for example, titanium dioxide, mica, iron oxides (for example Fe₂O₃, Fe₃O₄, FeO(OH)) and/or tin oxide. Advantageous dyes are, for example, carmine, Berlin Blue, Chromium Oxide Green, Ultramarine Blue and/or Manganese Violet. It is particularly advantageous to select the dyes and/or coloured pigments from the following list. The Colour Index numbers (CINs) are taken from the Rowe Colour Index, 3rd Edition, Society of Dyers and Colourists, Bradford, England, 1971.

| Chemical or other name | CIN | Colour |
|---|---|---|
| Pigment Green | 10006 | green |
| Acid Green 1 | 10020 | green |
| 2,4-Dinitrohydroxynaphthalene-7-sulfonic acid | 10316 | yellow |
| Pigment Yellow 1 | 11680 | yellow |
| Pigment Yellow 3 | 11710 | yellow |
| Pigment Orange 1 | 11725 | orange |
| 2,4-Dihydroxyazobenzene | 11920 | orange |
| Solvent Red 3 | 12010 | red |
| 1-(2′-Chloro-4′-nitro-1′-phenylazo)-2-hydroxynaphthalene | 12085 | red |
| Pigment Red 3 | 12120 | red |
| Ceres Red; Sudan Red; Fat Red G | 12150 | red |
| Pigment Red 112 | 12370 | red |
| Pigment Red 7 | 12420 | red |
| Pigment Brown 1 | 12480 | brown |
| N-(5-chloro-2,4-dimethoxyphenyl)-4-[[5-[(diethylamino)- | 12490 | red |
| sulfonyl]-2-methoxyphenyl]azo]-3-hydroxynaphthalene-2- | | |
| carboxamide | | |
| Disperse Yellow 16 | 12700 | yellow |
| 1-(4-Sulfo-1-phenylazo)-4-aminobenzene-5-sulfonic acid | 13015 | yellow |
| 2,4-Dihydroxy-azobenzene-4′-sulfonic acid | 14270 | orange |
| 2-(2,4-Dimethylphenylazo-5-sulfonyl)-1-hydroxynaphthalene- | 14700 | red |
| 4-sulfonic acid | | |
| 2-(4-Sulfo-1-naphthylazo)-1-naphthol-4-sulfonic acid | 14720 | red |
| 2-(6-Sulfo-2,4-xylylazo)-1-naphthol-5-sulfonic acid | 14815 | red |
| 1-(4′-Sulfophenylazo)-2-hydroxynaphthalene | 15510 | orange |
| 1-(2-Sulfonyl-4-chloro-5-carboxy-1-phenylazo)-2-hydroxy- | 15525 | red |
| naphthalene | | |
| 1-(3-Methylphenylazo-4-sulfonyl)-2-hydroxynaphthalene | 15580 | red |
| 1-(4′,(8′)-Sulfonyl)-2-hydroxynaphthalene | 15620 | red |
| 2-Hydroxy-1,2′-azonaphthalene-1′-sulfonic acid | 15630 | red |
| 3-Hydroxy-4-phenylazo-2-naphthylcarboxylic acid | 15800 | red |
| 1-(2-Sulfo-4-methyl-1-phenylazo)-2-naphthylcarboxylic acid | 15850 | red |
| 1-(2-Sulfo-4-methyl-5-chloro-1-phenylazo)-2-hydroxy- | 15865 | red |
| naphthalene-3-carboxylic acid | | |
| 1-(2-Sulfo-1-naphthylazo)-2-hydroxynaphthalene-3-carboxylic | 15880 | red |
| acid | | |
| 1-(3-Sulfo-1-phenylazo)-2-naphthol-6-sulfonic acid | 15980 | orange |
| 1-(4-Sulfo-1-phenylazo)-2-naphthol-6-sulfonic acid | 15985 | yellow |
| Allura Red | 16035 | red |
| 1-(4-Sulfo-1-naphthylazo)-2-naphthol-3,6-disulfonic acid | 16185 | red |
| Acid Orange 10 | 16230 | orange |
| 1-(4-Sulfo-1-naphthylazo)-2-naphthol-6,8-disulfonic acid | 16255 | red |
| 1-(4-Sulfo-1-naphthylazo)-2-naphthol-3,6,8-trisulfonic acid | 16290 | red |
| 8-Amino-2-phenylazo-1-naphthol-3,6-disulfonic acid | 17200 | red |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-Sulfo-1-phenylazo)-1-(4-sulfophenyl)-5-hydroxy- | 19140 | yellow |
| pyrazolone-3-carboxylic acid | | |
| Pigment Yellow 16 | 20040 | yellow |
| 2,6-(4′-Sulfo-2″,4″-dimethyl)bisphenylazo)-1,3-dihydroxy- | 20170 | orange |
| benzene | | |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″-Sulfo-1″-phenylazo)-7′-sulfo-1′-naphthylazo]-1- | 27755 | black |
| hydroxy-7-aminonaphthalene-3,6-disulfonic acid | | |
| 4-[4″-Sulfo-1″-phenylazo)-7′-sulfo-1′-naphthylazo]-1-hydroxy- | 28440 | black |
| 8-acetylaminonaphthalene-3,5-disulfonic acid | | |
| Direct Orange 34, 39, 44, 46, 60 | 40215 | orange |
| Food Yellow | 40800 | orange |
| trans-β-Apo-8′-carotene aldehyde (C₃₀) | 40820 | orange |
| trans-Apo-8′-carotinic acid (C₃₀) ethyl ester | 40850 | orange |
| Canthaxanthine | 40850 | orange |
| Acid Blue 1 | 42045 | blue |
| 2,4-Disulfo-5-hydroxy-4′-4″-bis(diethylamino)triphenylcarbinol | 42051 | blue |
| 4-[(-4-N-Ethyl-p-sulfobenzylamino)-phenyl-(4-hydroxy-2-sulfophenyl) | 42053 | green |
| (methylene)-1-(N-ethyl-N-p-sulfobenzyl)-2,5-cyclohexadienimine] | | |
| Acid Blue 7 | 42080 | blue |
| (N-Ethyl-p-sulfobenzylamino)phenyl-(2-sulfophenyl)methylene- | 42090 | blue |
| (N-ethyl-N-p-sulfobenzyl)-Δ^{2,5}-cyclohexadienimine | | |
| Acid Green 9 | 42100 | green |
| Diethyldisulfobenzyldi-4-amino-2-chlorodi-2-methylfuchsonimmonium | 42170 | green |
| Basic Violet 14 | 42510 | violet |
| Basic Violet 2 | 42520 | violet |
| 2′-Methyl-4′-(N-ethyl-N-m-sulfobenzyl)amino-4″-(N-diethyl)- | 42735 | blue |
| amino-2-methyl-N-ethyl-N-m-sulfobenzylfuchsonimmonium | | |
| 4′-(N-Dimethyl)amino-4″-(N-phenyl)aminonaphtho-N- | 44045 | blue |
| dimethylfuchsonimmonium | | |
| 2-Hydroxy-3,6-disulfo-4,4′-bisdimethylaminonaphthofuchsonimmonium | 44090 | green |
| Acid Red 52 | 45100 | red |
| 3-(2′-Methylphenylamino)-6-(2′-methyl-4′-sulfophenylamino)-9- | 45190 | violet |
| (2″-carboxyphenyl)xanthenium salt | | |
| Acid Red 50 | 45220 | red |
| Phenyl-2-oxyfluorone-2-carboxylic acid | 45350 | yellow |
| 4,5-Dibromofluorescein | 45370 | orange |
| 2,4,5,7-Tetrabromofluorescein | 45380 | red |
| Solvent Dye | 45396 | orange |
| Acid Red 98 | 45405 | red |
| 3′,4′,5′,6′-Tetrachloro-2,4,5,7-tetrabromofluorescein | 45410 | red |
| 4,5-Diiodofluorescein | 45425 | red |
| 2,4,5,7-Tetraiodofluorescein | 45430 | red |
| Quinophthalone | 47000 | yellow |
| Quinophthalonedisulfonic acid | 47005 | yellow |
| Acid Violet 50 | 50325 | violet |
| Acid Black 2 | 50420 | black |
| Pigment Violet 23 | 51319 | violet |
| 1,2-Dioxyanthraquinone, calcium aluminium complex | 58000 | red |
| 3-Oxypyrene-5,8,10-sulfonic acid | 59040 | green |
| 1-Hydroxy-4-N-phenylaminoanthraquinone | 60724 | violet |
| 1-Hydroxy-4-(4′-methylphenylamino)anthraquinone | 60725 | violet |
| Acid Violet 23 | 60730 | violet |
| 1,4-Di(4′-methylphenylamino)anthraquinone | 61565 | green |
| 1,4-Bis(o-sulfo-p-toluidino)anthraquinone | 61570 | green |
| Acid Blue 80 | 61585 | blue |
| Acid Blue 62 | 62045 | blue |
| N,N′-Dihydro-1,2,1′,2′-anthraquinonazine | 69800 | blue |
| Vat Blue 6; Pigment Blue 64 | 69825 | blue |
| Vat Orange 7 | 71105 | orange |
| Indigo | 73000 | blue |
| Indigodisulfonic acid | 73015 | blue |
| 4,4′-Dimethyl-6,6′-dichlorothioindigo | 73360 | red |
| 5,5′-Dichloro-7,7′-dimethylthioindigo | 73385 | violet |
| Quinacridone Violet 19 | 73900 | violet |
| Pigment Red 122 | 73915 | red |
| Pigment Blue 16 | 74100 | blue |
| Phthalocyanine | 74160 | blue |
| Direct Blue 86 | 74180 | blue |
| Chlorinated phthalocyanines | 74260 | green |
| Natural Yellow 6, 19; Natural Red 1 | 75100 | yellow |
| Bixin, Nor-Bixin | 75120 | orange |
| Lycopene | 75125 | yellow |
| Trans-alpha-, beta- or gamma-carotene | 75130 | orange |
| Keto and/or hydroxyl derivatives of carotene | 75135 | yellow |
| Guanine or pearlescent agent | 75170 | white |
| 1,7-Bis(4-hydroxy-3-methoxyphenyl)-1,6-heptadiene-3,5-dione | 75300 | yellow |
| Complex salt (Na, Al, Ca) of carminic acid | 75470 | red |
| Chlorophyll a and b; copper compounds of chlorophylls and | 75810 | green |
| chlorophyllines | | |
| Aluminium | 77000 | white |
| Aluminium hydroxide | 77002 | white |
| Water-containing aluminium silicates | 77004 | white |
| Ultramarine | 77007 | blue |
| Pigment Red 101 and 102 | 77015 | red |
| Barium sulfate | 77120 | white |
| Bismuth oxychloride and mixtures thereof with mica | 77163 | white |
| Calcium carbonate | 77220 | white |
| Calcium sulfate | 77231 | white |
| Carbon | 77266 | black |
| Pigment Black 9 | 77267 | black |
| Carbo medicinalis vegetabilis | 77268:1 | black |
| Chromium oxide | 77288 | green |
| Chromium oxide, water-containing | 77278 | green |
| Pigment Blue 28, Pigment Green 14 | 77346 | green |
| Pigment Metal 2 | 77400 | brown |
| Gold | 77480 | brown |
| Iron oxides and hydroxides | 77489 | orange |
| Iron oxide | 77491 | red |
| Iron oxide hydrate | 77492 | yellow |
| Iron oxide | 77499 | black |
| Mixtures of iron(II) and iron(III) hexacyanoferrate | 77510 | blue |
| Pigment White 18 | 77713 | white |
| Manganese ammonium diphosphate | 77742 | violet |
| Manganese phosphate; Mn₃(PO₄)₂.7H₂O | 77745 | red |
| Silver | 77820 | white |
| Titanium dioxide and mixtures thereof with mica | 77891 | white |
| Zinc oxide | 77947 | white |
| 6,7-Dimethyl-9-(1′-D-ribityl)isoalloxazine, lactoflavin | | yellow |
| Sugar dye | | brown |
| Capsanthin, capsorubin | | orange |
| Betanin | | red |
| Benzopyrylium salts, anthocyans | | red |
| Aluminium, zinc, magnesium and calcium stearate | | white |
| Bromothymol Blue | | blue |

It may furthermore be favourable to select, as dye, one or more substances from the following group:

2,4-dihydroxyazobenzene, 1-(2′-chloro-4′-nitro-1′-phenylazo)-2-hydroxynaphthalene, Ceres Red, 2-(4-sulfo-1-naphthylazo)-1-naphthol-4-sulfonic acid, the calcium salt of 2-hydroxy-1,2′-azonaphthalene-1′-sulfonic acid, the calcium and barium salts of 1-(2-sulfo-4-methyl-1-phenylazo)-2-naphthylcarboxylic acid, the calcium salt of 1-(2-sulfo-1-naphthylazo)-2-hydroxynaphthalene-3-carboxylic acid, the aluminium salt of 1-(4-sulfo-1-phenylazo)-2-naphthol-6-sulfonic acid, the aluminium salt of 1-(4-sulfo-1-naphthylazo)-2-naphthol-3,6-disulfonic acid, 1-(4-sulfo-1-naphthylazo)-2-naphthol-6,8-disulfonic acid, the aluminium salt of 4-(4-sulfo-1-phenylazo)-2-(4-sulfophenyl)-5-hydroxypyrazolone-3-carboxylic acid, the aluminium and zirconium salts of 4,5-dibromofluorescein, the aluminium and zirconium salts of 2,4,5,7-tetrabromofluorescein, 3′,4′,5′,6′-tetrachloro-2,4,5,7-tetrabromofluorescein and its aluminium salt, the aluminium salt of 2,4,5,7-tetraiodofluorescein, the aluminium salt of quinophthalonedisulfonic acid, the aluminium salt of indigodisulfonic acid, red and black iron oxide (CIN: 77 491 (red) and 77 499 (black)), iron oxide hydrate (CIN: 77492), manganese ammonium diphosphate and titanium dioxide.

Also advantageous are oil-soluble natural dyes, such as, for example, paprika extract, β-carotene or cochineal.

Also advantageous for the purposes of the present invention are gel creams comprising pearlescent pigments. Particular preference is given to the types of pearlescent pigment listed below:
1. Natural pearlescent pigments, such as, for example,
   a) “pearl essence” (guanine/hypoxanthine mixed crystals from fish scales) and
   b) “mother-of-pearl” (ground mussel shells)
2. Monocrystalline pearlescent pigments, such as, for example, bismuth oxychloride (BiOCl)
3. Layered substrate pigments: for example mica/metal oxide

The basis for pearlescent pigments is formed by, for example, pulverulent pigments or castor oil dispersions of bismuth oxychloride and/or titanium dioxide as well as bismuth oxychloride and/or titanium dioxide on mica. The lustre pigment listed under CIN 77163, for example, is particularly advantageous.

Also advantageous are, for example, the following pearlescent pigment types based on mica/metal oxide:

| Group | Coating/layer thickness | Colour |
|---|---|---|
| Silver-white pearlescent | TiO₂: 40-60 nm | silver |
| pigments | | |
| Interference pigments | TiO₂: 60-80 nm | yellow |
| | TiO₂: 80-100 nm | red |
| | TiO₂: 100-140 nm | blue |
| | TiO₂: 120-160 nm | green |
| Coloured lustre pigments | Fe₂O₃ | bronze |
| | Fe₂O₃ | copper |
| | Fe₂O₃ | red |
| | Fe₂O₃ | red-violet |
| | Fe₂O₃ | red-green |
| | Fe₂O₃ | black |
| Combination pigments | TiO₂/Fe₂O₃ | gold shades |
| | TiO₂/Cr₂O₃ | green |
| | TiO₂/Berlin Blue | dark blue |

Particular preference is given to, for example, the pearlescent pigments available from Merck under the trade names Timiron, Colorona or Dichrona.

The list of the said pearlescent pigments is of course not intended to be limiting. Pearlescent pigments which are advantageous for the purposes of the present invention can be obtained by numerous routes known per se. For example, other substrates apart from mica can also be coated with further metal oxides, such as, for example, silica and the like. For example, TiO₂- and Fe₂O₃-coated SiO₂ particles (“Ronasphere” grades), which are marketed by Merck and are particularly suitable for the optical reduction of fine wrinkles, are advantageous.

It may additionally be advantageous to completely omit a substrate such as mica. Particular preference is given to pearlescent pigments prepared using SiO₂. Such pigments, which may additionally also have goniochromatic effects, are available, for example, from BASF under the trade name Sicopearl Fantastico.

It may also be advantageous to employ Engelhard/Mearl pigments based on calcium sodium borosilicate coated with titanium dioxide. These are available under the name Reflecks. Due to their particle size of 40-80 μm, they have a glitter effect in addition to the colour.

Also particularly advantageous are effect pigments available from Flora Tech under the trade name Metasomes Standard/Glitter in various colours (yellow, red, green and blue). The glitter particles here are in the form of mixtures with various auxiliaries and dyes (such as, for example, the dyes with the colour index (CI) numbers 19140, 77007, 77289 and 77491).

The dyes and pigments can be in individual form or in the form of a mixture and mutually coated with one another, with different colour effects generally being caused by different coating thicknesses. The total amount of dyes and colouring pigments is advantageously selected from the range from, for example, 0.1% by weight to 30% by weight, preferably from 0.5 to 15% by weight, in particular from 1.0 to 10% by weight, in each case based on the total weight of the compositions.

Compositions according to the invention or compositions to be used in accordance with the invention may comprise at least one repellent, where the repellent is preferably selected from N,N-diethyl-3-methylbenzamide, ethyl 3-(acetylbutylamino)propionate, dimethyl phthalate, butopyronoxyl, 2,3,4,5-bis(2-butylene)tetrahydro-2-furaldehyde, N,N-diethylcaprylamide, N,N-diethylbenzamide, o-chloro-N,N-diethylbenzamide, dimethyl carbate, di-n-propyl isocinchomeronate, 2-ethylhexane-1,3-diol, N-octylbicycloheptenedicarboximide, piperonyl butoxide, 1-(2-methylpropoxycarbonyl)-2-(hydroxyethyl)piperidine, or mixtures thereof, where it is particularly preferably selected from N,N-diethyl-3-methylbenzamide, ethyl 3-(acetylbutylamino)propionate, 1-(2-methylpropoxycarbonyl)-2-(hydroxyethyl)piperidine, octanoic acid, decanoic acid, pyrethrins, pyrethroids, methyl nonyl ketone (undecan-2-one), cycloalkanecarboxylic acids, permethrin and (R)-p-mentha-1,8-diol, as well as effectivevderivatives of the said active ingredients which are known from the literature, or mixtures thereof.

The compositions according to the invention which comprise repellents are preferably insect repellents. Insect repellents are preferably available in the form of solutions, gels, sticks, rollers, pump sprays and aerosol sprays, with solutions and sprays forming the majority of the commercially available products. The basis for these two product forms are usually alcoholic or aqueous/alcoholic solutions with addition of fatting substances and slight perfuming. However, the compositions according to the invention may, in particular, also be emulsions or gels which comprise additional active ingredients oir whose principal purpose consists in another application, for example light protection or day care.

In a preferred embodiment of the present invention, the composition to be employed is a composition for the protection of body cells against oxidative stress, in particular for reducing skin ageing, characterised in that it comprises one or more antioxidants besides the said substances.

There are many proven substances known from the specialist literature which can be used as antioxidants, for example amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine) in very low tolerated doses (for example pmol to μmol/kg), and also (metal) chelating agents (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), vitamin A and derivatives (for example vitamin A palmitate), and coniferyl benzoate of benzoin resin, rutinic acid and derivatives thereof, α-glycosyl rutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Mixtures of antioxidants are likewise suitable for use in the cosmetic compositions according to the invention. Known and commercial mixtures are, for example, mixtures comprising, as active ingredients, lecithin, L-(+)-ascorbyl palmitate and citric acid (for example Oxynex® AP), natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004).

The compositions according to the invention may comprise vitamins as further ingredients. The cosmetic compositions according to the invention preferably comprise vitamins and vitamin derivatives selected from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active ingredient), thiamine (vitamin B₁), nicotinic acid (niacin), pyridoxine, pyridoxal, pyridoxamine (vitamin B₆), pantothenic acid, biotin, folic acid and cobalamine (vitamin B₁₂), particularly preferably vitamin A palmitate, vitamin C and derivatives thereof, DL-α-tocopherol, tocopherol E acetate, nicotinic acid, pantothenic acid and biotin.

Of the phenols having an antioxidative action, the polyphenols, some of which are naturally occurring, are of particular interest for applications in the pharmaceutical, cosmetic or nutrition sector. For example, the flavonoids or bioflavonoids, which are principally known as plant dyes, frequently have an antioxidant potential. K. Lemanska, H. Szymusiak, B. Tyrakowska, R. Zielinski, I. M. C. M. Rietjens; Current Topics in Biophysics 2000, 24 (2), 101-108, are concerned with effects of the substitution pattern of mono- and dihydroxyflavones. It is observed therein that dihydroxyflavones containing an OH group adjacent to the keto function or OH groups in the 3′,4′- or 6,7- or 7,8-position have antioxidative properties, while other mono- and dihydroxyflavones in some cases do not have antioxidative properties.

Quercetin (cyanidanol, cyanidenolon 1522, meletin, sophoretin, ericin, 3,3′,4′,5,7-pentahydroxyflavone) is frequently mentioned as a particularly effective antioxidant (for example C. A. Rice-Evans, N. J. Miller, G. Paganga, Trends in Plant Science 1997, 2 (4), 152-159). K. Lemanska, H. Szymusiak, B. Tyrakowska, R. Zielinski, A. E. M. F. Soffers, I. M. C. M. Rietjens; Free Radical Biology&Medicine 2001, 31 (7), 869-881, have investigated the pH dependence of the antioxidant action of hydroxyflavones. Quercetin exhibits the greatest activity amongst the structures investigated over the entire pH range.

Suitable antioxidants are furthermore compounds of the formula I 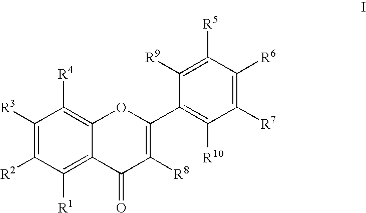
where R¹ to R¹⁰ may be identical or different and are selected from
   H
   OR¹¹
   straight-chain or branched C₁- to C₂₀-alkyl groups,
   straight-chain or branched C₃- to C₂₀-alkenyl groups,
   straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
   C₃- to C₁₀-cycloalkyl groups and/or C₃- to C₁₂-cycloalkenyl groups,
   where the rings may each also be bridged by —(CH₂)ₙ— groups,
   where n=1 to 3,
   where all OR¹¹ are, independently of one another,
      OH
      straight-chain or branched C₁- to C₂₀-alkoxy groups,
      straight-chain or branched C₃- to C₂₀-alkenyloxy groups,
      straight-chain or branched C₁- to C₂₀-hydroxyalkoxy groups, where the hydroxyl group(s) may be bonded to a primary or secondary carbon atom of the chain and furthermore the alkyl chain may also be interrupted by oxygen, and/or
      C₃- to C₁₀-cycloalkoxy groups and/or C₃- to C₁₂-cycloalkenyloxy groups, where the rings may each also be bridged by —(CH₂)ₙ— groups, where n=1 to 3, and/or
      mono- and/or oligoglycosyl radicals,
   with the proviso that at least 4 radicals from R¹ to R⁷ are OH and that at least 2 pairs of adjacent —OH groups are present in the molecule,
   or R², R⁵ and R⁶ are OH and the radicals R¹, R³, R⁴ and R⁷⁻¹⁰ are H,
      as described in German patent application DE-A 10244282.7.

Compositions which are particularly preferred in accordance with the invention may also serve for sun protection and then also comprise UV filters.

In principle, all UV filters are suitable for combination with the diffractive colorants to be employed in accordance with the invention. Particular preference is given to UV filters whose physiological acceptability has already been demonstrated. Both for UVA and UVB filters, there are many proven substances which are known from the specialist literature, for example
benzylidenecamphor derivatives, such as 3-(4′-methylbenzylidene)-dl-camphor (for example Eusolex® 6300), 3-benzylidenecamphor (for example Mexoryl® SD), polymers of N-{(2 and 4)-[(2-oxoborn-3-ylidene)methyl]-benzyl}acrylamide (for example Mexoryl® SW), N,N,N-trimethyl-4-(2-oxoborn-3-ylidenemethyl)anilinium methylsulfate (for example Mexoryl® SK) or (2-oxoborn-3-ylidene)toluene-4-sulfonic acid (for example Mexoryl® SL),
benzoyl- or dibenzoylmethanes, such as 1-(4-tert-butylphenyl)-3-(4-methoxyphenyl)propane-1,3-dione (for example Eusolex® 9020) or 4-isopropyldibenzoylmethane (for example Eusolex® 8020),
benzophenones, such as 2-hydroxy-4-methoxybenzophenone (for example Eusolex® 4360) or 2-hydroxy-4-methoxybenzophenone-5-sulfonic acid and its sodium salt (for example Uvinul® MS-40),
methoxycinnamic acid esters, such as octyl methoxycinnamate (for example Eusolex® 2292), isopentyl 4-methoxycinnamate, for example as a mixture of the isomers (for example Neo Heliopan® E 1000),
salicylate derivatives, such as 2-ethylhexyl salicylate (for example Eusolex® OS), 4-isopropylbenzyl salicylate (for example Megasol®) or 3,3,5-trimethylcyclohexyl salicylate (for example Eusolex® HMS),
4-aminobenzoic acid and derivatives, such as 4-aminobenzoic acid, 2-ethylhexyl 4-(dimethylamino)benzoate (for example Eusolex® 6007) or ethoxylated ethyl 4-aminobenzoate (for example Uvinul® P25),
phenylbenzimidazolesulfonic acids, such as 2-phenylbenzimidazole-5-sulfonic acid and the potassium, sodium and triethanolamine salts thereof (for example Eusolex® 232), 2,2-(1,4-phenylene)bisbenzimidazole-4,6-disulfonic acid and salts thereof (for example Neoheliopan® AP) or 2,2-(1,4-phenylene)bisbenzimidazole-6-sulfonic acid;
and further substances, such as
   2-ethylhexyl 2-cyano-3,3-diphenylacrylate (for example Eusolex® OCR),
   3,3′-(1,4-phenylenedimethylene)bis(7,7-dimethyl-2-oxobicyclo[2.2.1]-hept-1-ylmethanesulfonic acid and salts thereof (for example Mexoryl® SX),
   2,4,6-trianilino-(p-carbo-2′-ethylhexyl-1′-oxy)-1,3,5-triazine (for example Uvinul® T 150) and
   hexyl 2-(4-diethylamino-2-hydroxybenzoyl)benzoate (for example Uvinul® UVA Plus, BASF).

The compounds mentioned in the list should only be regarded as examples. It is of course also possible to use other UV filters.

These organic UV filters are generally incorporated into the formulations in an amount of from 0.5 to 10 percent by weight, preferably 1-8%.

Further suitable organic UV filters are, for example,
2-(2H-benzotriazol-2-yl)-4-methyl-6-(2-methyl-3-(1,3,3,3-tetra methyl-1-(trimethylsilyloxy)disiloxanyl)propyl)phenol (for example Silatrizole®),
2-ethylhexyl 4,4′-[(6-[4-((1,1-dimethylethyl)aminocarbonyl)phenylamino]-1,3,5-triazine-2,4-diyl)diimino]bis(benzoate) (for example Uvasorb® HEB),
α-(trimethylsilyl)-ω-[trimethylsilyl)oxy]poly[oxy(dimethyl [and approx. 6% of methyl[2-[p-[2,2-bis(ethoxycarbonyl]vinyl]phenoxy]-1-methylenethyl] and approx. 1.5% of methyl[3-[p-[2,2-bis(ethoxycarbonyl)vinyl)phenoxy)propenyl) and 0.1 to 0.4% of (methylhydrogen]silylene]] (n˜60) (CAS No. 207 574-74-1)
2,2′-methylenebis(6-(2H-benzotriazol-2-yl)-4-(1,1,3,3-tetramethylbutyl)phenol) (CAS No. 103 597-45-1)
2,2′-(1,4-phenylene)bis(1H-benzimidazole-4,6-disulfonic acid, monosodium salt) (CAS No. 180 898-37-7) and
2,4-bis{[4-(2-ethylhexyloxy)-2-hydroxyl]phenyl}-6-(4-methoxyphenyl)-1,3,5-triazine (CAS No. 103 597-45-, 187 393-00-6).

Further suitable UV filters are also methoxyflavones corresponding to the earlier German patent application DE-A-10232595.

Organic UV filters are generally incorporated into cosmetic formulations in an amount of from 0.5 to 20 percent by weight, preferably 1-15%.

Conceivable inorganic UV filters are those from the group consisting of the titanium dioxides, such as, for example, coated titanium dioxide (for example Eusolex® T-2000, Eusolex® T-AQUA, Eusolex® T-AVO), zinc oxides (for example Sachtotec®), iron oxides and also cerium oxides. These inorganic UV filters are generally incorporated into cosmetic compositions in an amount of from 0.5 to 20 percent by weight, preferably 2-10%.

Preferred compounds having UV-filtering properties are 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazole-5-sulfonic acid and the potassium, sodium and triethanolamine salts thereof.

Optimised compositions may comprise, for example, the combination of the organic UV filters 4′-methoxy-6-hydroxyflavone with 1-(4-tert-butylphenyl)-3-(4-methoxyphenyl)propane-1,3-dione and 3-(4′-methylbenzylidene)-dl-camphor. This combination gives rise to broad-band protection, which can be supplemented by the addition of inorganic UV filters, such as titanium dioxide microparticles.

All the said UV filters can also be employed in encapsulated form. In particular, it is advantageous to employ organic UV filters in encapsulated form. In detail, the following advantages arise:
The hydrophilicity of the capsule wall can be set independently of the solubility of the UV filter. Thus, for example, it is also possible to incurporate hydrophobic UV filters into purely aqueous compositions. In addition, the oily impression on application of the composition comprising hydrophobic UV filters, which is frequently regarded as unpleasant, is suppressed.
Certain UV filters, in particular dibenzoylmethane derivatives, exhibit only reduced photostability in cosmetic compositions. Encapsulation of these filters or compounds which impair the photostability of these filters, such as, for example, cinnamic acid derivatives, enables the photostability of the entire composition to be increased.
Skin penetration by organic UV filters and the associated potential for irritation on direct application to the human skin is repeatedly being discussed in the literature. The encapsulation of the corresponding substances which is proposed here suppresses this effect.
In general, encapsulation of individual UV filters or other ingredients enables preparation problems caused by the interaction of individual composition constituents with one another, such as crystallisation processes, precipitation and agglomerate formation, to be avoided since the interaction is suppressed.

It is therefore preferred in accordance with the invention for one or more of the above-mentioned UV filters to be in encapsulated form. It is advantageous here for the capsules to be so small that they cannot be viewed with the naked eye. In order to achieve the above-mentioned effects, it is furthermore necessary for the capsules to be sufficiently stable and the encapsulated active ingredient (UV filter) only to be released to the environment to a small extent, or not at all.

Suitable capsules can have walls of inorganic or organic polymers. For example, U.S. Pat. No. 6,242,099 B1 describes the production of suitable capsules with walls of chitin, chitin derivatives or polyhydroxylated polyamines. Capsules which can particularly preferably be employed in accordance with the invention have walls which can be obtained by a sol-gel process, as described in the applications WO 00/09652, WO 00/72806 and WO 00/71084. Preference is again given here to capsules whose walls are built up from silica gel (silica; undefined silicon oxide hydroxide). The production of corresponding capsules is known to the person skilled in the art, for example from the cited patent applications, whose contents expressly also belong to the subject-matter of the present application.

The capsules in compositions according to the invention are preferably present in amounts which ensure that the encapsulated UV filters are present in the composition in the above-indicated amounts.

The compositions according to the invention may in addition comprise further conventional skin-protecting or skin-care active ingredients. These may in principle be any active ingredients known to the person skilled in the art.

Particularly preferred active ingredients are, for example, also so-called solutes. These are substances which are involved in the osmoregulation of plants or microorganisms and can be isolated from these organisms. The generic term compatible solutes here also encompasses the osmolytes described in German Patent Application DE-A-10133202. Suitable osmolytes are, for example, the polyols, methylamine compounds and amino acids and respective precursors thereof. Osmolytes in the sense of German Patent Application DE-A-10133202 are, in particular, substances from the group consisting of the polyols, such as, for example, myo-inositol, mannitol or sorbitol, and/or one or more of the osmolytically active substances mentioned below:

taurine, choline, betaine, phosphorylcholine, glycerophosphorylcholines, glutamine, glycine, α-alanine, glutamate, aspartate and proline. Precursors of these substances are, for example, glucose, glucose polymers, phosphatidylcholine, phosphatidylinositol, inorganic phosphates, proteins, peptides and polyamino acids. Precursors are, for example, compounds which are converted into osmolytes by metabolic steps.

Compatible solutes which are preferably employed in accordance with the invention are substances selected from the group consisting of pyrimidinecarboxylic acids (such as ectoin and hydroxyectoin), proline, betaine, glutamine, cyclic diphosphoglycerate, N-acetylornithine, trimethylamine N-oxide, di-myo-inositol phosphate (DIP), cyclic 2,3-diphosphoglycerate (cDPG), 1,1-diglycerol phosphate (DGP), β-mannosyl glycerate (firoin), β-mannosyl glyceramide (firoin-A) and/or dimannosyl diinositol phosphate (DMIP) or an optical isomer, derivative, for example an acid, a salt or ester of these compounds, or combinations thereof.

Of the pyrimidinecarboxylic acids, particular mention should be made here of ectoin ((S)-1,4,5,6-tetrahydro-2-methyl-4-pyrimidinecarboxylic acid) and hydroxyectoin ((S,S)-1,4,5,6-tetrahydro-5-hydroxy-2-methyl-4-pyrimidinecarboxylic acid) and derivatives thereof. These compounds stabilise enzymes and other biomolecules in aqueous solutions and organic solvents. Furthermore, they stabilise, in particular, enzymes against denaturing conditions, such as salts, extreme pH values, surfactants, urea, guanidinium chloride and other compounds.

Ectoin and ectoin derivatives, such as hydroxyectoin, can advantageously be used in medicaments. In particular, hydroxyectoin can be employed for the preparation of a medicament for the treatment of skin diseases. Other areas of application of hydroxyectoin and other ectoin derivatives are typically in areas in which, for example, trehalose is used as additive. Thus, ectoin derivatives, such as hydroxyectoin, can be used as protectant in dried yeast and bacteria cells. Pharmaceutical products, such as non-glycosylated, pharmaceutically active peptides and proteins, for example t-PA, can also be protected with ectoin or its derivatives.

Of the cosmetic applications, particular mention should be made of the use of ectoin and ectoin derivatives for the care of aged, dry or irritated skin.

Thus, European patent application EP-A-0 671 161 describes, in particular, that ectoin and hydroxyectoin are employed in cosmetic compositions, such as powders, soaps, surfactant-containing cleansing products, lipsticks, rouge, make-up, care creams and sunscreen preparations.

Preference is given here to the use of a pyrimidinecarboxylic acid of the following formula II 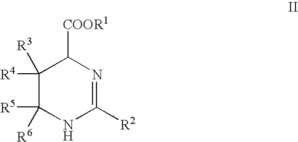
in which R¹ is a radical H or C1-8-alkyl, R² is a radical H or C1-4-alkyl, and R³, R⁴, R⁵ and R⁶ are each, independently of one another, a radical from the group consisting of H, OH, NH₂ and C1-4-alkyl. Preference is given to the use of pyrimidinecarboxylic acids in which R² is a methyl or ethyl group, and R¹ or R⁵ and R⁶ are H. Particular preference is given to the use of the pyrimidinecarboxylic acids ectoin ((S)-1,4,5,6-tetrahydro-2-methyl-4-pyrimidinecarboxylic acid) and hydroxyectoin ((S,S)-1,4,5,6-tetrahydro-5-hydroxy-2-methyl-4-pyrimidinecarboxylic acid). In this case, the compositions according to the invention preferably comprise pyrimidinecarboxylic acids of this type in amounts of up to 15% by weight.

Particular preference is given in accordance with the invention to the selection of the compatible solutes from di-myo-inositol phosphate (DIP), cyclic 2,3-diphosphoglycerate (cDPG), 1,1-diglycerol phosphate (DGP), β-mannosyl glycerate (firoin), β-mannosyl glyceramide (firoin-A) and/or dimannosyl diinositol phosphate (DMIP), ectoin, hydroxyectoin or mixtures thereof.

Of the aryl oximes likewise preferably employed, preference is given to the use of 2-hydroxy-5-methyllaurophenone oxime, which is also known as HMLO, LPO or F5. Its suitability for use in cosmetic compositions is disclosed, for example, in DE-A-41 16 123. Compositions which comprise 2-hydroxy-5-methyllaurophenone oxime are accordingly suitable for the treatment of skin diseases which are accompanied by inflammation. It is known that compositions of this type can be used, for example, for the therapy of psoriasis, various forms of eczema, irritative and toxic dermatitis, UV dermatitis and further allergic and/or inflammatory diseases of the skin and integumentary appendages. Compositions according to the invention which, in addition to the compound of the formula I, additionally comprise an aryl oxime, preferably 2-hydroxy-5-methyllaurophenone oxime, exhibit surprising antiinflamematory suitability. The compositions here preferably comprise from 0.01 to 10% by weight of the aryl oxime, it being particularly preferred for the composition to comprise from 0.05 to 5% by weight of aryl oxime.

In a further, likewise preferred embodiment of the present invention, the composition according to the invention comprises at least one self-tanning agent.

Advantageous self-tanning agents which can be employed are, inter alia:

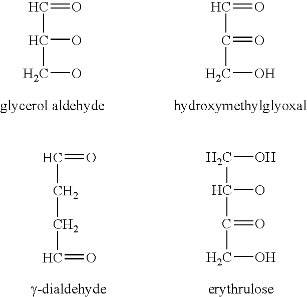

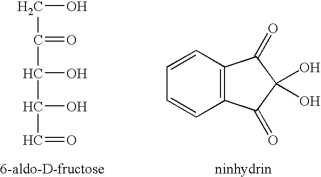

Mention should also be made of 5-hydroxy-1,4-naphthoquinone (juglone), which is extracted from the shells of fresh walnuts 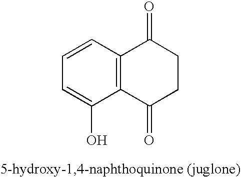
and 2-hydroxy-1,4-naphthoquinone (lawsone), which occurs in henna leaves

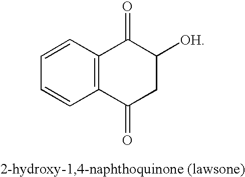

Very particular preference is given to 1,3-dihydroxyacetone (DHA), a trifunctional sugar which occurs in the human body, and derivatives thereof.

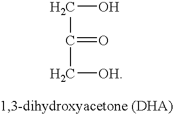

All compounds or components which can be used in the compositions are either known or commercially available or can be synthesised by known processes.

The diffractive colorants, effect pigments and further active ingredients can be incorporated into cosmetic or dermatological compositions in the customary manner. Suitable compositions are those for external use, for example in the form of a cream, lotion or gel or as a solution which can be sprayed onto the skin. Suitable for internal use are administration forms such as capsules, coated tablets, powders, tablet solutions or solutions.

Use forms of the compositions according to the invention that may be mentioned are, for example, solutions, suspensions, emulsions, PIT emulsions, pastes, ointments, gels, creams, lotions, powders, soaps, surfactant-containing cleansing preparations, oils, aerosols and sprays. Examples of other use forms are sticks, shampoos and shower compositions. Any desired customary carriers, assistants and, if desired, further active ingredients may be added to the composition.

Preferred assistants originate from the group consisting of preservatives, antioxidants, stabilisers, solubilisers, vitamins, colorants and odour improvers.

Ointments, pastes, creams and gels may comprise the customary carriers, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary carriers, for example lactose, talc, silica, aluminium hydroxide, calcium silicate and polyamide powder, or mixtures of these substances. Sprays may additionally comprise the customary propellants, for example chlorofluorocarbons, propane/butane or dimethyl ether.

Solutions and emulsions may comprise the customary carriers, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

Suspensions may comprise the customary carriers, such as liquid diluents, for example water, ethanol or propylene glycol, suspending agents, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary carriers, such as alkali metal salts of fatty acids, salts of fatty acid monoesters, fatty acid protein hydrolysates, isethionates, lanolin, fatty alcohol, vegetable oils, plant extracts, glycerol, sugars, or mixtures of these substances.

Surfactant-containing cleansing products may comprise the customary carriers, such as salts of fatty alcohol sulfates, fatty alcohol ether sulfates, sulfosuccinic acid monoesters, fatty acid protein hydrolysates, isethionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary carriers, such as synthetic oils, such as fatty acid esters, fatty alcohols, silicone oils, natural oils, such as vegetable oils and oily plant extracts, paraffin oils or lanolin oils, or mixtures of these substances.

Further typical cosmetic use forms are also lipsticks, lip-care sticks, mascara, eyeliner, eye-shadow, rouge, powder make-up, emulsion make-up and wax make-up, and sunscreen, pre-sun and after-sun preparations.

The preferred composition forms according to the invention also include, in particular, emulsions.

Emulsions according to the invention are advantageous and comprise, for example, the said fats, oils, waxes and other fatty substances, as well as water and an emulsifier, as usually used for a composition of this type.

The lipid phase may advantageously be selected from the following group of substances:
mineral oils, mineral waxes;
oils, such as triglycerides of capric or caprylic acid, furthermore natural oils, such as, for example, castor oil;
fats, waxes and other natural and synthetic fatty substances, preferably esters of fatty acids with alcohols having a low carbon number, for example with isopropanol, propylene glycol or glycerol, or esters of fatty alcohols with alkanoic acids having a low carbon number or with fatty acids;
silicone oils, such as dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously selected from the group consisting of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, or from the group consisting of esters of aromatic carboxylic acids and saturated and/or unsaturated, branched and/or unbranched alcohols having a chain length of from 3 to 30 carbon atoms. Ester oils of this type can then advantageously be selected from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyidodecyl palmitate, oleyl oleate, oleyl erucate, erucyl oleate, erucyl erucate and synthetic, semi-synthetic and natural mixtures of esters of this type, for example jojoba oil.

The oil phase may furthermore advantageously be selected from the group consisting of branched and unbranched hydrocarbons and waxes, silicone oils, dialkyl ethers, or the group consisting of saturated and unsaturated, branched and unbranched alcohols, and fatty acid triglycerides, specifically the triglycerol esters of saturated and/or unsaturated, branched and/or unbranched alkanecarboxylic acids having a chain length of from 8 to 24, in particular 12-18, carbon atoms. The fatty acid triglycerides may advantageously be selected, for example, from the group consisting of synthetic, semi-synthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed for the purposes of the present invention. It may also be advantageous to employ waxes, for example cetyl palmitate, as the only lipid component of the oil phase.

The oil phase is advantageously selected from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric acid triglyceride and dicapryl ether.

Particularly advantageous are mixtures of C₁₂₋₁₅-alkyl benzoate and 2-ethylhexyl isostearate, mixtures of C₁₂₋₁₅-alkyl benzoate and isotridecyl isononanoate, as well as mixtures of C₁₂₋₁₅-alkyl benzoate, 2-ethylhexyl isostearate and isotridecyl isononanoate.

Of the hydrocarbons, paraffin oil, squalane and squalene may advantageously be used for the purposes of the present invention.

Furthermore, the oil phase may also advantageously have a content of cyclic or linear silicone oils or consist entirely of oils of this type, although it is preferred to use an additional content of other oil-phase components in addition to the silicone oil or the silicone oils.

The silicone oil to be used in accordance with the invention is advantageously cyclomethicone (octamethylcyclotetrasiloxane). However, it is also advantageous for the purposes of the present invention to use other silicone oils, for example hexamethylcyclotrisiloxane, polydimethylsiloxane or poly(methylphenylsiloxane).

Also particularly advantageous are mixtures of cyclomethicone and isotridecyl isononanoate and of cyclomethicone and 2-ethylhexyl isostearate.

The aqueous phase of the compositions according to the invention optionally advantageously comprises alcohols, diols or polyols having a low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low carbon number, for example ethanol, isopropanol, 1,2-propanediol or glycerol, and, in particular, one or more thickeners, which may advantageously be selected from the group consisting of silicon dioxide, aluminium silicates, polysaccharides and derivatives thereof, for example hyaluronic acid, xanthan gum, hydroxypropylmethylcellulose, particularly advantageously from the group consisting of the polyacrylates, preferably a polyacrylate from the group consisting of the so-called Carbopols, for example Carbopol grades 980, 981, 1382, 2984 or 5984, in each case individually or in combination.

In particular, mixtures of the above-mentioned solvents are used. In the case of alcoholic solvents, water may be a further constituent.

Emulsions according to the invention are advantageous and comprise, for example, the said fats, oils, waxes and other fatty substances, as well as water and an emulsifier, as usually used for a formulation of this type.

In a preferred embodiment, the compositions according to the invention comprise hydrophilic surfactants.

The hydrophilic surfactants are preferably selected from the group consisting of the alkylglucosides, acyl lactylates, betaines and coconut amphoacetates.

The alkylglucosides are themselves advantageously selected from the group consisting of the alkylglucosides which are distinguished by the structural formula 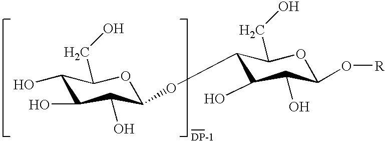
where R is a branched or unbranched alkyl radical having from 4 to 24 carbon atoms, and where \overline{DP} denotes a mean degree of glucosylation of up to 2.

The value \overline{DP} represents the degree of glucosidation of the alkylglucosides used in accordance with the invention and is defined as

$$\overline{D P} = \frac{{p}_{1}}{100} \cdot 1 + \frac{{p}_{2}}{100} \cdot 2 + \frac{{p}_{3}}{100} \cdot 3 + … = \sum \frac{{p}_{i}}{100} \cdot i$$

in which p₁, p₂, p₃ . . . pᵢ represent the proportion of mono-, di-, tri- . . . i-fold glucosylated products in percent by weight. Products which are advantageous according to the invention are those having degrees of glucosylation of 1-2, particularly advantageously of from 1.1 to 1.5, very particularly advantageously of 1.2-1.4, in particular of 1.3.

The value DP takes into account the fact that alkylglucosides are generally, as a consequence of their preparation, in the form of mixtures of mono- and oligoglucosides. A relatively high content of monoglucosides, typically in the order of 40-70% by weight, is advantageous in accordance with the invention.

Alkylglycosides which are particularly advantageously used for the purposes of the invention are selected from the group consisting of octyl glucopyranoside, nonyl glucopyranoside, decyl glucopyranoside, undecyl glucopyranoside, dodecyl glucopyranoside, tetradecyl glucopyranoside and hexadecyl glucopyranoside.

It is likewise advantageous to employ natural or synthetic raw materials and assistants or mixtures which are distinguished by an effective content of the active ingredients used in accordance with the invention, for example Plantaren® 1200 (Henkel KGaA), Oramix® NS 10 (Seppic).

The acyllactylates are themselves advantageously selected from the group consisting of the substances which are distinguished by the structural formula 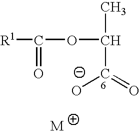
where R¹ is a branched or unbranched alkyl radical having from 1 to 30 carbon atoms, and M⁺ is selected from the group consisting of the alkali metal ions and the group consisting of ammonium ions which are substituted by one or more alkyl and/or one or more hydroxyalkyl radicals, or corresponds to half an equivalent of an alkaline earth metal ion.

For example, sodium isostearyl lactylate, for example the product Pathionic® ISL from the American Ingredients Company, is advantageous.

The betaines are advantageously selected from the group consisting of the substances which are distinguished by the structural formula 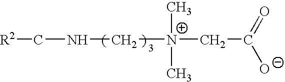
where R² is a branched or unbranched alkyl radical having from 1 to 30 carbon atoms.

R² is particularly advantageously a branched or unbranched alkyl radical having from 6 to 12 carbon atoms.

For example, capramidopropylbetaine, for example the product Tego® Betain 810 from Th. Goldschmidt AG, is advantageous.

A coconut amphoacetate which is advantageous for the purposes of the invention is, for example, sodium coconut amphoacetate, as available under the name Miranol® Ultra C32 from Miranol Chemical Corp.

The compositions according to the invention are advantageously characterised in that the hydrophilic surfactant(s) is (are) present in concentrations of 0.01-20% by weight, preferably 0.05-10% by weight, particularly preferably 0.1-5% by weight, in each case based on the total weight of the composition.

For use, the cosmetic and dermatological compositions are applied in sufficient amount to the skin and/or hair in the usual manner for cosmetics.

Cosmetic and dermatological compositions according to the invention may exist in various forms. Thus, they may be, for example, a solution, a water-free composition, an emulsion or microemulsion of the water-in-oil (W/O) or oil-in-water (O/W) type, a multiple emulsion, for example of the water-in-oil-in-water (W/O/W) type, a gel, a solid stick, an ointment or an aerosol. It is also advantageous to administer ectoin in encapsulated form, for example in collagen matrices and other conventional encapsulation materials, for example as cellulose encapsulations, in gelatine, wax matrices or liposomally encapsulated. In particular, wax matrices, as described in DE-A 43 08 282, have proven favourable. Preference is given to emulsions. O/W emulsions are particularly preferred. Emulsions, W/O emulsions and O/W emulsions are obtainable in a conventional manner.

Emulsifiers that can be used are, for example, the known W/O and O/W emulsifiers. It is advantageous to use further conventional co-emulsifiers in the preferred O/W emulsions according to the invention.

Co-emulsifiers which are advantageous according to the invention are, for example, O/W emulsifiers, principally from the group consisting of the substances having HLB values of 11-16, very particularly advantageously having HLB values of 14.5-15.5, so long as the O/W emulsifiers have saturated radicals R and R′. If the O/W emulsifiers have unsaturated radicals R and/or R′ or in the case of isoalkyl derivatives, the preferred HLB value of such emulsifiers may also be lower or higher.

It is advantageous to select the fatty alcohol ethoxylates from the group consisting of ethoxylated stearyl alcohols, cetyl alcohols, cetylstearyl alcohols (cetearyl alcohols). Particular preference is given to the following: polyethylene glycol (13) stearyl ether (steareth-13), polyethylene glycol (14) stearyl ether (steareth-14), polyethylene glycol (15) stearyl ether (steareth-15), polyethylene glycol (16) stearyl ether (steareth-16), polyethylene glycol (17) stearyl ether (steareth-17), polyethylene glycol (18) stearyl ether (steareth-18), polyethylene glycol (19) stearyl ether (steareth-19), polyethylene glycol (20) stearyl ether (steareth-20), polyethylene glycol (12) isostearyl ether (isosteareth-12), polyethylene glycol (13) isostearyl ether (isosteareth-13), polyethylene glycol (14) isostearyl ether (isosteareth-14), polyethylene glycol (15) isostearyl ether (isosteareth-15), polyethylene glycol (16) isostearyl ether (isosteareth-16), polyethylene glycol (17) isostearyl ether (isosteareth-17), polyethylene glycol (18) isostearyl ether (isosteareth-18), polyethylene glycol (19) isostearyl ether (isosteareth-19), polyethylene glycol (20) isostearyl ether (isosteareth-20), polyethylene glycol (13) cetyl ether (ceteth-13), polyethylene glycol (14) cetyl ether (ceteth-14), polyethylene glycol (15) cetyl ether (ceteth-15), polyethylene glycol (16) cetyl ether (ceteth-16), polyethylene glycol (17) cetyl ether (ceteth-17), polyethylene glycol (18) cetyl ether (ceteth-18), polyethylene glycol (19) cetyl ether (ceteth-19), polyethylene glycol (20) cetyl ether (ceteth-20), polyethylene glycol (13) isocetyl ether (isoceteth-13), polyethylene glycol (14) isocetyl ether (isoceteth-14), polyethylene glycol (15) isocetyl ether (isoceteth-15), polyethylene glycol (16) isocetyl ether (isoceteth-16), polyethylene glycol (17) isocetyl ether (isoceteth-17), polyethylene glycol (18) isocetyl ether (isoceteth-18), polyethylene glycol (19) isocetyl ether (isoceteth-19), polyethylene glycol (20) isocetyl ether (isoceteth-20), polyethylene glycol (12) oleyl ether (oleth-12), polyethylene glycol (13) oleyl ether (oleth-13), polyethylene glycol (14) oleyl ether (oleth-14), polyethylene glycol (15) oleyl ether (oleth-15), polyethylene glycol (12) lauryl ether (laureth-12), polyethylene glycol (12) isolauryl ether (isolaureth-12), polyethylene glycol (13) cetylstearyl ether (ceteareth-13), polyethylene glycol (14) cetylstearyl ether (ceteareth-14), polyethylene glycol (15) cetylstearyl ether (ceteareth-15), polyethylene glycol (16) cetylstearyl ether (ceteareth-16), polyethylene glycol (17) cetylstearyl ether (ceteareth-17), polyethylene glycol (18) cetylstearyl ether (ceteareth-18), polyethylene glycol (19) cetylstearyl ether (ceteareth-19), polyethylene glycol (20) cetylstearyl ether (ceteareth-20).

It is furthermore advantageous to select the fatty acid ethoxylates from the following group:

polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

An ethoxylated alkyl ether carboxylic acid or salt thereof which can advantageously be used is sodium laureth-11 carboxylate. An alkyl ether sulfate which can advantageously be used is sodium laureth-14 sulfate. An ethoxylated cholesterol derivative which can advantageously be used is polyethylene glycol (30) cholesteryl ether. Polyethylene glycol (25) soyasterol has also proven successful. Ethoxylated triglycerides which can advantageously be used are the polyethylene glycol (60) evening primrose glycerides.

It is furthermore advantageous to select the polyethylene glycol glycerol fatty acid esters from the group consisting of polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/caprinate, polyethylene glycol (20) glyceryl oleate, polyethylene glycol (20) glyceryl isostearate, polyethylene glycol (18) glyceryl oleate/cocoate.

It is likewise favourable to select the sorbitan esters from the group consisting of polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

Optional W/O emulsifiers, but ones which may nevertheless be advantageous for the purposes of the invention can be the following:

fatty alcohols having from 8 to 30 carbon atoms, monoglycerol esters of saturated and/or unsaturated, branched and/or unbranched alkanecarboxylic acids having a chain length of from 8 to 24 carbon atoms, in particular 12-18 carbon atoms, diglycerol esters of saturated and/or unsaturated, branched and/or unbranched alkanecarboxylic acids having a chain length of from 8 to 24 carbon atoms, in particular 12-18 carbon atoms, monoglycerol ethers of saturated and/or unsaturated, branched and/or unbranched alcohols having a chain length of from 8 to 24 carbon atoms, in particular 12-18 carbon atoms, diglycerol ethers of saturated and/or unsaturated, branched and/or unbranched alcohols having a chain length of from 8 to 24 carbon atoms, in particular 12-18 carbon atoms, propylene glycol esters of saturated and/or unsaturated, branched and/or unbranched alkanecarboxylic acids having a chain length of from 8 to 24 carbon atoms, in particular 12-18 carbon atoms, and sorbitan esters of saturated and/or unsaturated, branched and/or unbranched alkanecarboxylic acids having a chain length of from 8 to 24 carbon atoms, in particular 12-18 carbon atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate and glyceryl monocaprylate.

Preferred compositions according to the invention are particularly suitable for protecting human skin against ageing processes and against oxidative stress, i.e. against damage by free radicals, as are produced, for example, by sunlight, heat or other influences. In this connection, they are in the various administration forms usually used for this application. For example, they may, in particular, be in the form of a lotion or emulsion, such as in the form of a cream or milk (O/W, W/O, O/W/O, W/O/W), in the form of oily-alcoholic, oily-aqueous or aqueous-alcoholic gels or solutions, in the form of solid sticks or may be formulated as an aerosol.

The composition may comprise cosmetic adjuvants which are usually used in this type of composition, such as, for example, thickeners, softeners, moisturisers, surfactants, emulsifiers, preservatives, antifoams, perfumes, waxes, lanolin, propellants, dyes and/or pigments which colour the composition itself or the skin, and other ingredients usually used in cosmetics.

The dispersant or solubiliser used can be an oil, wax or other fatty substance, a lower monoalcohol or lower polyol or mixtures thereof. Particularly preferred monoalcohols or polyols include ethanol, isopropanol, propylene glycol, glycerol and sorbitol.

A preferred embodiment of the invention is an emulsion in the form of a protective cream or milk which comprises, for example, fatty alcohols, fatty acids, fatty acid esters, in particular triglycerides of fatty acids, lanolin, natural and synthetic oils or waxes and emulsifiers in the presence of water.

Further preferred embodiments are oily lotions based on natural or synthetic oils and waxes, lanolin, fatty acid esters, in particular triglycerides of fatty acids, or oily-alcoholic lotions based on a lower alcohol, such as ethanol, or a glycerol, such as propylene glycol, and/or a polyol, such as glycerol, and oils, waxes and fatty acid esters, such as triglycerides of fatty acids.

The composition according to the invention may also be in the form of an alcoholic gel which comprises one or more lower alcohols or polyols, such as ethanol, propylene glycol or glycerol, and a thickener, such as siliceous earth. The oily-alcoholic gels also comprise natural or synthetic oil or wax.

The solid sticks consist of natural or synthetic waxes and oils, fatty alcohols, fatty acids, fatty acid esters, lanolin and other fatty substances.

If a composition is formulated as an aerosol, the customary propellants, such as alkanes, fluoroalkanes and chlorofluoroalkanes, are generally used.

The cosmetic composition may also be used to protect the hair against photochemical damage in order to prevent changes of colour shade, bleaching or damage of a mechanical nature. In this case, a suitable formulation is in the form of a rinse-out shampoo, lotion, gel or emulsion, the composition in question being applied before or after shampooing, before or after colouring or bleaching or before or after permanent waving. It is also possible to select a composition in the form of a lotion or gel for styling or treating the hair, in the form of a lotion or gel for brushing or blow-waving, in the form of a hair lacquer, permanent waving composition, colorant or bleach for the hair. The composition may comprise various adjuvants used in this type of composition, such as surfactants, thickeners, polymers, softeners, preservatives, foam stabilisers, electrolytes, organic solvents, silicone derivatives, oils, waxes, antigrease agents, dyes and/or pigments which colour the composition itself or the hair, or other ingredients usually used for hair care.

The compositions according to the invention can be prepared here with the aid of techniques which are well known to the person skilled in the art.

If the diffractive colorants used in accordance with the invention are films, a preferred use is the use thereof as or in artificial nails. The present application therefore furthermore relates to an artificial nail comprising a diffractive colorant. The nail here may consist of the diffractive colorant film, or the diffractive colorant may form the decorative layer of an artificial nail.

Thus, for example, a film comprising diffractive colorants can, after cutting to match the size of the individual fingernail or toenail, be detached from the support sheet and applied to the fingernail/toenail and subsequently fixed using clear nail varnish. Alternatively, the films may themselves be provided with an adhesive layer, so that subsequent varnishing is unnecessary. Placing of the film in a varnish before application to the fingernail is also a variant of the present invention in which subsequent varnishing is unnecessary.

Furthermore, the diffractive colorants to be employed in accordance with the invention can also be employed in varnishes, such as UV-curable acrylic varnishes, which are used for the production of artificial nails.

A further use of the diffractive colorants which is preferred in accordance with the invention is use in nail-care compositions, in particular nail varnishes. It may be particularly preferred here for the nail-care composition only to comprise the above-described spheres or core/shell particles and the photonic structure which makes up the actual diffractive colorant not to form until during or after application of the varnish. In another, likewise preferred embodiment, however, the nail-care compositions may also comprise diffractive colorants directly, in particular in pigmentary form.

Nail-care compositions, in particular in the form of nail varnishes, are amongst the most-used decorative cosmetics. They usually comprise a synthetic resin as film former and inorganic or organic pigments or dyes. The nail varnishes are intended to exhibit high gloss, high hardness and good adhesion to keratin-containing substances, such as fingernails, and to dry rapidly at room temperature to give a non-tacky, uniform film. The high gloss and the good adhesion should be retained over the longest possible period. In order that the nail varnishes can be removed again using conventional nail varnish removers, the film-forming resins used must be soluble in water/acetone mixtures. By contrast, the film-forming resins should be insoluble in water or water/alcohol mixtures in order that the nail varnish is not dissolved on contact with water or during handling of conventional household chemicals.

Typical nail varnishes which are suitable for use in accordance with the invention essentially consist of solutions, suspensions or emulsions of certain substances. Particular mention should be made here of binders, such as nitrocellulose, or various synthetic resins, such as, in particular, polyacrylates, polymethacrylates, toluenesulfonamide-formaldehyde resins, toluenesulfonamide-epoxy resins, alkyd resins or polyvinyl acetate. Furthermore, nail varnishes usually comprise suitable solvents for the binders, with solvent mixtures usually being employed. Typical solvents, besides water, are alcohols, esters and ketones, such as, in particular, methyl, ethyl, propyl and butyl acetate, isopropyl alcohol and n-butyl alcohol. The nail varnishes may optionally be provided with further additives for forming the optical appearance, for example soluble dyes. In addition, the mixture may comprise plasticisers (such as, for example, camphor or dibutyl phthalate) and extenders (for example toluene or xylene). These nail varnishes cure through evaporation of the solvent and the other volatile constituents as a function of the layer thickness of the varnish, the temperature and the type of solvent. In addition, the curing time is also dependent on other influences (for example air flow).

Particular preference is given in accordance with the invention to water-based nail-care compositions which preferably comprise polyurethanes or polyacrylates as binder.

Thus, EP-A-0 424 112 describes nail-varnish formulations which comprise an emulsion polymer having a core/shell structure as binder. The polymer of the outer shell has a softening temperature which is lower than that of the polymer of the inner shell. DE-19727504 discloses aqueous cosmetic formulations, in particular nail-varnish formulations, which comprise, as binder, an emulsion polymer which is obtainable by polymerisation of a mixture of certain monomers in the presence of a polymer containing monomers containing an ionic or ionogenic group.

Besides water, water-based nail varnishes comprise water-soluble resins, such as, for example, polyurethane resins, acrylic resins, alkyd resins, epoxy resins or melamine resins. The resin here is preferably selected in such a way that it is water-soluble, appears white or transparent in water, is inexpensive and simple to process, is as far as possible non-combustible and is not toxic or hyperallergenic. It may furthermore be essential that the varnish dries rapidly and has good film-forming properties at temperatures from 10° C.

In a particularly preferred variant of the invention, the diffractive colorant or the precursor of the diffractive colorant simultaneously serves as binder in the nail-care composition. The nail-care composition here preferably comprises no further binder, and the precursor of the diffractive colorant is preferably core/shell particles as already described above. Particular preference is given here to core/shell particles having a polyacrylate shell or polyurethane shell. Corresponding core/shell particles that are preferably to be employed are, as already stated above, described, in particular, in European patent application EP-A-0 955 323 or international patent application WO 03/25035.

Even without further comments, it is assumed that a person skilled in the art will be able to utilise the above description in its broadest scope. The preferred embodiments should therefore merely be regarded as descriptive disclosure which is absolutely not limiting in any way. The complete disclosure content of all applications and publications mentioned above and below is incorporated into this application by way of reference. The following examples are intended to illustrate the present invention. However, they should in no way be regarded as limiting. All compounds or components which can be used in the compositions are either known and commercially available or can be synthesised by known methods. The INCI names of the raw materials used are indicated (the INCI names are by definition given in English).

### EXAMPLES

Abbreviations used:
ALMA allyl methacrylate
AN acrylonitrile
APS ammonium peroxodisulfate
BDDA butane-1,4-diol diacrylate
CHMA cyclohexyl methacrylate
MMA methyl methacrylate
SDS dodecyl sulfate sodium salt
SDTH sodium dithionite
PCHMA poly(cyclohexyl methacrylate)
PS poly(styrene)

### Example 1

### Production of Spheres as Primary Units

### Example 1a

### Production of Monodisperse Silicon Dioxide Spheres Having a Diameter of 250 nm

1140 ml of ethanol and 430 ml of demineralised water are introduced into a flask. The mixture is heated to 63° C. with gentle stirring. 266 ml of 25% ammonia solution are then added. 168 ml of tetraethyl orthosilicate (likewise at a temperature of 63° C.) are added in one portion and rapidly with vigorous stirring. After stirring for a further 15 seconds, the stirrer is switched off, and the dispersion is left to stand for 18 h. Evaporation of the dispersion is followed by azeotropic distillation in order to remove the ethanol and the ammonia, so that the SiO₂ spheres are in a purely aqueous dispersion.

### Example 1b

### Production of Monodisperse PMMA Spheres Having a Diameter of 300 nm

A round-bottomed flask is charged with 540 ml of distilled water and 100 ml of methyl methacrylate. After heating to 80° C., a nitrogen gas blanket is introduced onto the dispersion. The impeller stirrer is set to 300 rpm, and 0.5 g of 2,2′-azobis(2-methylpropionamidine) dihydrochloride is then added. After 2 h, the reaction is complete, and the flask contents are cooled. The product is purified by repeated centrifugation and decantation.

### Example 2

### Organisation of the Spheres in a Colloid-Crystalline Packing (Opal Packing)

The dispersions from Examples 1a and 1b are filtered with suction via a Buchner funnel with fine filter paper via a water-pump vacuum. Initially, the colloidal spheres pass through the suction filter (evident from the cloudy filtrate), but partial blockage of the filter pores occurs rapidly, so that only the water is sucked through and the spheres remain as filter cake. The regular arrangement of the spheres generates shimmering colour effects.

### Example 3

### Production of Opal Colour Particles

The shimmering filter cake produced from SiO₂ colloid spheres in Example 2 starting from Example 1a is carefully heated to 400° C. with the filter paper in an oven for crosslinking of the SiO₂ spheres. After cooling, the shimmering material is removed and carefully comminuted, giving shimmering opal colour particles.

### Example 4

### Production of Inverse Opal Colour Particles

The shimmering filter cake produced from PMMA colloid spheres in Example 2 starting from Example 1b is impregnated with a precursor solution consisting of 7 ml of tetraethoxysilane, 4.7 ml of ethanol, 1 ml of conc. HCl and 3 ml of water with gentle vacuum suction. The charged filter cake is heated to 500° C. in an oven over a period of 10 h. During this time, the PMMA spheres are pyrolytically degraded, leaving shimmering coloured powders of inverse opals comprising SiO₂.

### Example 5

### Production of Particles Having a Polystyrene Core, p(MMA-co-ALMA) Interlayer and poly(cyclohexyl methacrylate) Shell

A mixture, held at a temperature of 4° C., consisting of 217 g of water, 3.6 g of styrene (Merck, destabilised) and 130 mg of SDS (Merck) is introduced into a 1 l stirred-tank reactor, pre-heated to 75° C., with propeller stirrer, argon protective-gas inlet and reflux condenser and dispersed with vigorous stirring. Immediately after the introduction, the reaction is initiated by directly successive addition of 50 mg of SDTH (Merck), 350 mg of APS (Merck) and a further 50 mg of SDTH, in each case dissolved in 5 g of water. After 20 min, a monomer emulsion comprising 8.1 g of BDDA (Merck, destabilised), 72.9 g of styrene (Merck, destabilised), 0.375 g of SDS, 0.1 g of KOH and 110 g of water is metered in continuously over a period of 120 min. The reactor contents are stirred for 30 min without further addition. 200 mg of APS, dissolved in 5 g of water, are then added. After stirring for a further 60 min, a second monomer emulsion comprising 1.5 g of ALMA (Merck, destabilised), 13.5 g of MMA (Merck, destabilised), 0.075 g of SDS and 20 g of water is metered in continuously over a period of 25 min. The reactor contents are subsequently stirred for 30 min without further addition. 200 mg of APS, dissolved in 5 g of water, are then added.

A monomer emulsion comprising 120 g of CHMA (Degussa, destabilised), 120 g of water and 0.4 g of SDS is subsequently metered in continuously over a period of 150 min. For virtually complete reaction of the monomers, the mixture is subsequently stirred for a further 60 min. The core/shell particles are subsequently precipitated in 1 l of methanol, the precipitation is completed by addition of 25 g of concentrated aqueous sodium chloride solution, 1 l of dist. water is added to the suspension, the mixture is filtered with suction, and the solid dried.

### Example 6

### Production of Particles Having a poly(methyl methacrylate) Core and Polystyrene Shell

A mixture, held at a temperature of 4° C., consisting of 217 g of water, 0.4 g of ALMA (Merck, destabilised), 3.6 g of MMA (Merck, destabilised) and 23 mg of SDS (Merck) is introduced into a 1 l stirred-tank reactor, preheated to 75° C., with propeller stirrer, argon protective-gas inlet and reflux condenser and dispersed with vigorous stirring. Immediately after the introduction, the reaction is initiated by directly successive addition of 30 mg of SDTH (Merck), 150 mg of APS (Merck) and a further 30 mg of SDTH, in each case dissolved in 5 g of water. After 20 min, a monomer emulsion comprising 9.6 g of ALMA (Merck, destabilised), 96 g of MMA (Merck, destabilised), 0.35 g of SDS, 0.1 g of KOH and 130 g of water is metered in continuously over a period of 120 min. The reactor contents are stirred for 60 min without further addition. 100 mg of APS, dissolved in 5 g of water, are then added. After stirring for a further 10 min, a second monomer emulsion comprising 120 g of styrene (Merck, destabilised), 0.4 g of SDS and 120 g of water is metered in continuously over a period of 150 min. For virtually complete reaction of the monomers, the mixture is subsequently stirred for a further 60 min. The core/shell particles are subsequently precipitated in 1 l of methanol, the precipitation is completed by addition of 25 g of concentrated aqueous sodium chloride solution, 1 l of dist. water is added to the suspension, the mixture is filtered with suction, and the solid dried.

### Example 7

### Production of Particles by Isostatic Pressing and Subsequent Grinding

5 g of the dried coagulate from Example 5 or 6 are compounded with 0.2% by weight of Licolub Fal and 0.2% by weight of Licolub WE 40 (both from Clariant) in a microextruder (Thermo Haake, Minilab 5, co-rotating screws) at a temperature of 190° C. and a screw speed of 200 rpm for 30 min. The extrudate from the perforated die is rolled up into a coil and pressed between two PET films using a laboratory press (Collin) in accordance with the following programme to give thin films:

Step

| | 1 | 2 | 3 | 4 |
|---|---|---|---|---|
| Pressing time | 3 | 3 | 1 | 8 |
| [min] | | | | |
| Pressure | 100 | 250 | 250 | 200 |
| [bar] | | | | |
| Temp. [° C.] | 180 | 180 | 130 | 20 |

The thin films are then ground in an agate mortar to give small particles.

### Example 8

### Preparation of a Dispersion

Formulations consisting of in each case 5% by weight of diffractive colorant from Example 3, 4 or 7 in the thixotropic nail varnish Base 1348 (International Lacquers S.A.) are prepared by stirring the pigment particles into the varnish.

The resultant dispersion is spread onto a paint card. Drying and curing of the varnish formulation gives a colour-effect coating comprising pale-reflecting pigments which have an intense green or intense blue colour, depending on the viewing direction.

### Example 9

### Production of Particles by Isostatic Pressing and Subsequent Grinding

The particles from Example 8 are converted into pigments analogously to Example 7.

### Example 10

### Production of Core/Shell Particles Having a Crosslinkable Shell

A mixture, held at a temperature of 4° C., consisting of 217 g of water, 0.4 g of butanediol diacrylate (Merck, destabilised), 3.6 g of styrene (BASF, destabilised) and 80 mg of sodium dodecylsulfate (SDS; Merck) is introduced into a 1 l stirred-tank reactor, pre-heated to 75° C., with propeller stirrer, argon protective-gas inlet and reflux condenser and dispersed with vigorous stirring. Immediately after the introduction, the reaction is initiated by directly successive addition of 50 mg of sodium dithionite (Merck), 250 mg of ammonium peroxodisulfate (Merck) and a further 50 mg of sodium dithionite (Merck), in each case dissolved in 5 g of water. After 10 min, a monomer emulsion comprising 6.6 g of butanediol diacrylate (Merck, destabilised), 59.4 g of styrene (BASF, destabilised), 0.3 g of SDS, 0.1 g of KOH and 90 g of water is metered in continuously over a period of 210 min. The reactor contents are stirred for 30 min without further addition. A second monomer emulsion comprising 3 g of allyl methacrylate (Merck, destabilised), 27 g of methyl methacrylate (BASF, destabilised), 0.15 g of SDS (Merck) and 40 g of water is subsequently metered in continuously over a period of 90 min. The reactor contents are subsequently stirred for 30 min without further addition. A monomer emulsion comprising 130 g of ethyl acrylate (BASF, destabilised), 139 g of water, 4 g of hydroxyethyl methacrylate and 0.33 g of SDS (Merck) is subsequently metered in continuously over a period of 180 min. For virtually complete reaction of the monomers, the mixture is subsequently stirred for a further 60 min. The core/shell particles are subsequently precipitated in 1 l of methanol, 1 l of dist. water is added, the mixture is filtered with suction, and the solid is dried.

### Example 10a

Core/shell particles whose shell consists of 80% by weight of cyclohexyl methacrylate, 18% by weight of ethyl acrylate and 2% by weight of hydroxyethyl methacrylate are produced analogously.

### Example 11

### Production of Particles by Extrusion of Films and Subsequent Grinding

3 kg of the core/shell particles from Example 10 are comminuted in a cutting mill (Rapid, model: 1528) with ice cooling and subsequently compounded with 2% by weight of isocyanate curing agent (Crelan™; Bayer) in a single-screw extruder (Plasti-Corder; Brabender; screw diameter 19 mm with 1-hole die (3 mm)). After a cooling zone, the mixture is granulated in an A 90-5 granulator (Automatik). The granules are processed in a flat-film unit consisting of a single-screw extruder (Göttfert; model: Extrusiometer; screw diameter 20 mm; L/D 25), a thickness-adjustable film die (width 135 mm) and a temperature-controllable smoothing stack (Leistritz; roll diameter 15 mm; roll width 350 mm). A film ribbon with a width of 125 mm and a thickness of 1 mm is obtained. The film is subsequently heated to 190° C. After crosslinking has taken place, the brittle film is comminuted in a roll mill to give pigment particles.

### Example 11a

### Production of Particles by Isostatic Pressing and Subsequent Grinding

The particles from Example 10a are converted into pigments analogously to Example 7.

### Example 12

### Production of Opal Colour Particles

The shimmering filter cake produced from SiO₂ colloid spheres in Example 2 starting from Example 1a is carefully impregnated with a molten wax (carnauba wax). After cooling, the shimmering material is removed and carefully comminuted, giving shimmering opal colour particles.

### Example 13

### Production of Opal Colour Particles

The shimmering filter cake produced from PMMA colloid spheres in Example 2 starting from Example 1b is carefully impregnated with a molten lanolin (Satulan®; Croda Oleo Chemicals Ltd). After cooling, the shimmering material is removed and carefully comminuted, giving shimmering opal colour particles.

### Example 14

### Production of Inverse Opal Colour Particles by Etching

### Example 14a

### Functionalisation of the SiO₂ Cores from Example 1

3 ml of MPS dissolved in ethanol are added to 1.3 l of ethanolic suspension comprising 2.5% by weight of SiO₂ (SiO₂ suspension having a violet dry colour (wavelength maximum l111=400 nm, average particle diameter according to TEM 201 nm; according to Example 1), 0.69 M of NH₃ and 2 M of H₂O with stirring at room temperature. The mixture is slowly warmed to 65° C. in a rotary evaporator, initially under atmospheric pressure. After 1.5 h, distillation of an azeotropic mixture of ethanol and water is commenced by reducing the pressure. The liquid distilled off is replaced with absolute ethanol. In total, 1.2 l of ethanol/water mixture are removed. After 2 h, the reaction solution is evaporated to 300 ml and transferred into a 1 l round-bottomed flask. 0.06 g of SDS, dissolved in 120 g of water, is added, and ethanol is again distilled off at 65° C. The liquid distilled off is replaced with water.

### Example 14b

### Emulsion Polymerisation

The emulsion polymerisation is carried out in a double-walled 250 ml glass reactor, thermostatted at 75° C., with inert-gas inlet, propeller stirrer and reflux condenser. Argon is bubbled through 110 g (comprising 17 g of SiO₂) of SiO₂ suspension in accordance with Example 2 for 20 min. 0.1 g of SDS is then added, and the mixture is introduced into the reactor. 0.05 g of SPS, dissolved in 3 g of water, is subsequently added. After 15 min, a monomer emulsion comprising 5.4 g of MMA, 0.6 g of ALMA, 0.02 g of SDS (0.33% by weight, based on the monomer), 0.04 g of KOH and 30 g of water is metered in continuously over a period of 90 min. The reactor contents are stirred for 20 min without further addition. 0.02 g of APS, dissolved in 3 g of water, is then added. After 10 min, a second monomer emulsion comprising 20 g of CHMA, 0.08 g of SDS (0.4% by weight, based on the monomer) and 40 g of water is metered in continuously over a period of 200 min. For virtually complete reaction of the monomers, the mixture is subsequently stirred for a further 120 min. The core/shell particles are subsequently precipitated in 500 ml of ethanol, the precipitation is completed by addition of 15 g of concentrated aqueous sodium chloride solution, 500 ml of dist. water are added to the suspension, the mixture is filtered with suction, and the polymer is dried at 50° C. under reduced pressure.

### Example 14c

### Production of the Template Film

The dried, pulverulent polymers from Example 3 are granulated at 200° C. in an extruder (microextruder from DSM Research). The granules are warmed in a hydraulic press (Collin 300 P) and pressed at a pre-specified hydraulic pressure. Flat metal plates covered with PET film are used as mould. A typical pressing programme for the production of films having a diameter of about 10 cm and a thickness of about 0.15 mm is:
Initial weight 2-3 g of polymer;
Prewarming for 5 min at 180° C., no pressure;
Pressing for 3 min at 180° C. at a hydraulic pressure of 1 bar;
Pressing for 3 min at 180° C. at a hydraulic pressure of 150 bar;
Slow cooling for 10 min at a hydraulic pressure of 150 bar, reaching about 90° C.;
Rapid cooling to room temperature, no pressure.

### Example 14d

### Etching of the Films Using Hydrofluoric Acid

The films are covered with hydrofluoric acid (10% by weight) in open vessels and exposed at RT for one week. Evaporating hydrofluoric acid is replaced with fresh. After rinsing with water and drying, the etched film pieces exhibit clearly recognisable reflection colours.

Investigation of ultrathin sections (100 nm) of the films after etching confirms that the SiO₂ cores are dissolved out of the films with retention of the order, and films with ordered porosity are formed.

### Example 14e

### Grinding of the Porous Films

Grinding of the films with ordered porosity gives shimmering inverse opal powders.

### Example 15

### Production of Inverse Opal Colour Particles by UV Degradation

Preparation of a Latex PTBMAcsPS

50 mg of sodium bisulfite, dissolved in 5 g of water, are admixed with an emulsion, held at a temperature of 4° C., consisting of 217 g of water, 0.4 g of ALMA, 3.6 g of TBMA and 30 mg of SDS, and the emulsion is transferred into the reactor pre-heated to 75° C. Immediately after the introduction, the reaction is initiated by addition of 220 mg of sodium peroxodisulfate and a further 50 mg of sodium bisulfite, each dissolved in 5 g of water. After 20 min, monomer emulsion I comprising 9.6 g of ALMA, 96 g of TBMA, 0.45 g of SDS, 0.1 g of KOH and 130 g of water is metered in continuously over a period of 180 min. The reactor contents are stirred for 30 min without further addition. 150 mg of sodium peroxodisulfate, dissolved in 5 g of water, are then added. After stirring for 15 min, monomer emulsion II comprising 120 g of styrene, 0.4 g of SDS and 120 g of water is metered in continuously over a period of 200 min. For virtually complete reaction of the monomers, the mixture is subsequently stirred for a further 60 min. Dried samples of the latex exhibit a green colour. Electron microscopic investigation of precipitates of the latex shows that the polymer particles have an irregular shape and an average particle size of about 210 nm. The core/shell particles are subsequently precipitated in 1 litre of ethanol, the precipitation is completed by addition of 25 g of concentrated aqueous sodium chloride solution, 1 litre of dist. water is added to the suspension, the mixture is filtered with suction, and the polymer is dried at 50° C. under reduced pressure.

Further latices having a larger particle diameter and more uniform particle shape are prepared by changing the type and concentration of the emulsifier:

| | Example | | | | | | | |
|---|---|---|---|---|---|---|---|---|
| | 15a | | 15b | | 15c | | 15d | |
| Starting mixture | | | | | | | | |
| Water | 217 | g | 217 | g | 217 | g | 217 | g |
| ALMA | 0.4 | g | 0.4 | g | 0.4 | g | 0.4 | g |
| TBMA | 3.6 | g | 3.6 | g | 3.6 | g | 3.6 | g |
| SDS | 0.03 | g | 0.03 | g | 0.03 | g | 0.03 | g |
| Initiation | | | | | | | | |
| Sodium bisulfite | 0.05 | g | 0.05 | g | 0.05 | g | 0.05 | g |
| Sodium peroxo- | 0.22 | g | 0.2 | g | 0.2 | g | 0.2 | g |
| disulfate | | | | | | | | |
| Sodium bisulfite | 0.05 | g | 0.05 | g | 0.05 | g | 0.05 | g |
| Emulsion I | | | | | | | | |
| Water | 130 | g | 130 | g | 130 | g | 130 | g |
| TBMA | 96 | g | 96 | g | 96 | g | 96 | g |
| ALMA | 9.6 | g | 9.6 | g | 9.6 | g | 9.6 | g |
| SDS | 0.45 | g | 0.4 | g | 0.4 | g | 0.4 | g |
| Triton X 405 | | | 0.2 | g | 0.2 | g | 0.2 | g |
| KOH | 0.1 | g | 0.1 | g | 0.1 | g | 0.1 | g |
| Initiator | | | | | | | | |
| Sodium peroxo- | 0.15 | g | 0.1 | g | 0.1 | g | 0.1 | g |
| disulfate | | | | | | | | |
| Emulsion II | | | | | | | | |
| Water | 120 | g | 140 | g | 140 | g | 140 | g |
| Styrene | 120 | g | 120 | g | 120 | g | 120 | g |
| SDS | 0.4 | g | 0.4 | g | 0.4 | g | 0.4 | g |
| Triton X405 | | | 0.3 | g | 0.3 | g | 0.3 | g |

| | | | | |
|---|---|---|---|---|
| Colour | green | red | green | reddish |
| | | (sec. planes) | (sec. planes) | |

Pressing of the Films

For the production of films, the polymer powders from Examples 15a-15d are warmed in a hydraulic press (Collin 300 P), and the melt is pressed at a pre-specified hydraulic pressure. Flat metal plates covered with PET film are used as mould. A typical pressing programme for the production of films having a diameter of about 10 cm and a thickness of about 0.2 mm is:
Initial weight 1-2 g of polymer;
Prewarming for 5 min at 180° C., no pressure;
Pressing for 3 min at 180° C. at a hydraulic pressure of 1 bar;
Pressing for 3 min at 180° C. at a hydraulic pressure of 150 bar;
Slow cooling for 10 min at a hydraulic pressure of 150 bar, reaching about 90° C.;
Rapid cooling to room temperature, no pressure.
   Production of Mouldings Having Homogeneous, Regularly Arranged Cavities

The films are laid beneath a UV lamp (high-pressure Hg vapour lamp, power 300 watts, lamp-film distance: 20 cm) for a period of 24 h. After the UV exposure, the films exhibit bright, iridescent colour effects.

Grinding of the Porous Films

Grinding of the films with ordered porosity gives shimmering inverse opal powders.

### Example 16

### Production of Inverse Opal Colour Particles by Calcination

A mixture, held at a temperature of 4° C., consisting of 1519 g of demineralised water, 2.8 g of 1,4-butanediol diacrylate (Merck), 25.2 g of styrene (Merck) and 1030 mg of sodium dodecylsulfate (Merck) is introduced into a 5 l double-jacket reactor, held at a temperature of 75° C., with double-propeller stirrer, argon protective-gas inlet and reflux condenser and dispersed with vigorous stirring.

Immediately thereafter, the reaction is initiated by successive injection of 350 mg of sodium dithionite (Merck), 1.75 g of ammonium peroxodisulfate (Merck) and a further 350 mg of sodium dithionite (Merck), each dissolved in about 20 ml of water. The injection is carried out by means of disposable syringes.

After 20 min, a monomer emulsion consisting of 56.7 g of 1,4-butanediol diacrylate (Merck), 510.3 g of styrene (Merck), 2.625 g of sodium dodecylsulfate (Merck), 0.7 g of KOH and 770 g of water is metered in continuously via the rotary piston pump over a period of 120 min.

The reactor contents are stirred for 30 min without further addition.

A second monomer emulsion consisting of 10.5 g of allyl methacrylate (Merck), 94.50 g of methyl methacrylate (Merck), 0.525 g of sodium dodecylsulfate (Merck) and 140 g of water is subsequently metered in continuously via the rotary piston pump over a period of 30 min.

After about 15 min, 350 mg of ammonium peroxodisulfate (Merck) are added, and the mixture is then stirred for a further 15 min.

Finally, a third monomer emulsion consisting of 200 g of ethyl acrylate (Merck), 0.550 g of sodium dodecylsulfate (Merck) and 900 g of water is metered in continuously via the rotary piston pump over a period of 240 min. The mixture is subsequently stirred for a further 120 min. Before and after each introduction of monomer emulsions and after introduction of the starting mixture, argon is passed into the double-jacket reactor as protective-gas blanket for about one minute.

Next day, the reactor is warmed to 95° C., and a steam distillation is carried out in order to remove residual unreacted monomers from the latex dispersion.

A dispersion of core/shell particles is obtained in which the shell has a proportion by weight of about 22%. The polystyrene core is crosslinked, and the interlayer is likewise crosslinked (p(MMA-co-ALMA)) and serves for grafting of the shell of uncrosslinked ethyl acrylate.

In order to form the template-forming structure, i.e. the organisation of the core/shell particles in close sphere packing, 5 g of the latex dispersion are poured into a flat glass dish having a diameter of 7 cm and dried in air, giving flakes which shimmer in colour.

One such flake is evacuated in a round-bottomed flask using a rotary slide-valve oil pump. A precursor solution consisting of 5 ml of tetra-n-butyl orthotitanate in 5 ml of absolute ethanol is subsequently added in a static vacuum, so that the dissolved precursor, driven by capillary forces, is able to penetrate into the cavities of the template. An argon blanket is added above the solution containing the impregnated template. This arrangement is left at a stand for a few hours before the impregnated flake is removed in a stream of argon protective gas and calcined at 500° C. in a corundum boat in a tubular furnace. Opalescent flakes are obtained.

### Example 17

### Production of Inverse Opal Colour Particles by Pyrolysis of Polymer Films

Preparation of the Core/Shell Latices PMMA-PSAN₅₀ (Shell Comprising 50% by Weight of Styrene and 50% by Weight of Acrylonitrile)

30 mg of sodium dithionite (SDTH, Merck), dissolved in 5 g of water, are admixed with a starting emulsion, held at a temperature of 4° C., consisting of 217 g of water, 0.4 g of allyl methacrylate (ALMA, Merck), 3.6 g of methyl methacrylate (MMA, Merck) and 20.5 mg of sodium dodecylsulfate (SDS, Merck).

The emulsion is transferred into a 1 l double-jacket stirred-tank reactor, held at a temperature of 75° C., with reflux condenser, argon gas inlet and double-propeller stirrer.

Immediately after introduction of the emulsion, the reaction is initiated by addition of 150 mg of ammonium peroxodisulfate (APS, Merck) and a further 30 mg of sodium dithionite (SDTH, Merck), each dissolved in 5 g of water.

After 20 min, a monomer emulsion consisting of 9.6 g of ALMA (Merck), 96 g of MMA (Merck), 0.35 g of SDS (Merck), 0.1 g of KOH (Merck) and 130 g of water is metered in continuously via a rotary piston pump over a period of 120 min.

The reactor contents are stirred for 60 min without further addition. 100 mg of APS (Merck), dissolved in 5 g of water, are then added.

After stirring for a further 10 min, a second monomer emulsion consisting of 60 g of styrene (Merck), 60 g of acrylonitrile, 0.33 g of SDS (Merck) and 120 g of water is metered in continuously via a rotary piston pump over a period of 160 min.

For virtually complete reaction of the monomers, the mixture is subsequently stirred for a further 60 min.

The core/shell particles are subsequently coagulated in 1 l of methanol, the precipitation is completed by addition of 25 g of concentrated aqueous sodium chloride solution, 1 l of dist. water is added to the suspension, the mixture is filtered with suction, and the polymeric coagulate is dried at 50° C. under reduced pressure.

An average particle size of the particles of 263 nm is determined with the aid of a transmission electron microscope.

Preparation of the Core/Shell Latices PMMA-PSAN₇₀ (Shell Comprising 30% by Weight of Styrene and 70% by Weight of Acrylonitrile)

Formulation see above, with the following differences:

The starting emulsion comprises 22 mg of SDS (Merck), the second monomer emulsion consists of 36 g of styrene (Merck), 84 g of acrylonitrile, 120 g of water, 0.4 g of SDS (Merck) and 0.34 g of Triton X405™.

Further Conversion of the Coagulate into Films

The coagulate consisting of PMMA-PSAN₅₀ latex particles is converted into a polymer extrudate in a DSM microextruder at 220° C. in a nitrogen atmosphere, and this is chopped to give pellets with a length of 5 mm. The pellets are pressed to give films.

The pressing of in each case 1-2 g of coagulate or pellets to give films is carried out under the following conditions in a Collin 300 P laboratory press:
prewarming for 5 min at 180° C., no pressure;
pressing for 3 min at 1 bar and 180° C.;
pressing for 3 min at 150 bar and 180° C.;
slow cooling for 10 min at 150 bar, reaching about 90° C.;
rapid cooling to room temperature, no pressure.

The films obtained have a thickness of about 0.2 mm, have an angle-dependent yellow-green colour when viewed perpendicularly and are viscoelastic.

Pyrolysis of the Films

Variant a:

The films are pyrolysed in an air atmosphere at 240° C. in a muffle furnace for 5 h.

The pyrolysed films have a black base colour on which a violet reflection colour is superimposed when viewed perpendicularly.

Variant b:

The films are conditioned in an air atmosphere at 200° C. for 2 weeks. The conditioned films, in which the polymer cores are still present, have a brown base colour on which a green reflection colour is superimposed when viewed perpendicularly.

The films are subsequently pyrolysed in an air atmosphere at 240° C. in a muffle furnace for 5 h.

The pyrolysed films have a black base colour, on which a violet reflection colour is superimposed when viewed perpendicularly. The pores in the film have a virtually spherical shape.

Grinding of the Porous Films

Grinding of the films with ordered porosity gives shimmering inverse opal powders.

### Formulation Examples

The pigment employed in the following formulation examples is in each case the photonic effect materials selected from one or more of the above Examples 3, 4, 7, 9, 11, 12, 13, 14, 15, 16 and 17.

In the formulations, in each case the trade name, the INCI name, and the proportion in % by weight and a source of supply for the raw materials are indicated.

### Example A

### Wet Lip Balm Comprising 10% of Diffractive Colorant

Phase A

| | | |
|---|---|---|
| Pigment | 10.00% | (1) |

Phase B

| | | | |
|---|---|---|---|
| Vegelatum Equiline EU103 | | 11.00% | (2) |
| Candelilla wax | Candelilla Cera | 7.00% | (3) |
| | (Candelilla Wax) | | |
| Beeswax, bleached | Cera Alba (Beeswax) | 5.00% | (1) |
| Myritol 331 | Cocoglycerides | 8.00% | (4) |
| Ozokerite wax white # 77W | Ozokerite | 3.00% | (3) |
| Isopropyl myristate | Isopropyl Myristate | 5.00% | (4) |
| Eusolex ® 2292 | Ethylhexyl | 7.00% | (1) |
| | Methoxycinnamate, BHT | | |
| Eusolex ® OCR | Octocrylene | 4.00% | (1) |
| Oxynex ® K liquid | PEG-8, Tocopherol, | 0.10% | (1) |
| | Ascorbyl Palmitate, | | |
| | Ascorbic Acid, Citric Acid | | |
| Propyl 4-hydroxybenzoate | Propylparaben | 0.10% | (1) |
| Colour dispersion in castor oil | | 0.50% | |
| Castor oil | Ricinus Communis | 38.80% | (5) |
| | (Castor Oil) | | |

Phase C

| | | | |
|---|---|---|---|
| Tendresse # 75418C | Parfum | 0.50% | (6) |
| perfume oil | | | |

Preparation:

The constituents of phase B are heated at 75° C. with stirring until everything has melted. Add phase A and stir well. Then introduce the lipstick composition into the casting apparatus held at a temperature of 65° C., add phase C, and stir for 15 minutes. The homogeneous melt is poured into the casting mould pre-warmed to 55° C.

The moulds are subsequently cooled, and the castings are removed when cold. After warming of the lipsticks to room temperature, the lipsticks are briefly flame-treated.

Sources of Supply:

(1) Merck KGaA/Rona®
(2) Natunola Health Inc.
(3) Ross Waxes
(4) Cognis GmbH
(5) Henry Lamotte GmbH
(6) Haarmann & Reimer GmbH

### Example B

### Shower Gel

Phase A

| | | | |
|---|---|---|---|
| Pigment | | 0.10% | (1) |
| Keltrol T | Xanthan Gum | 0.75% | (2) |
| Water, demineralised | Aqua (Water) | 64.95% | |

Phase B

| | | | |
|---|---|---|---|
| Plantacare 2000 UP | Decyl Glucoside | 20.00% | (3) |
| Texapon ASV 50 | Sodium Laureth Sulfate, | 3.60% | (3) |
| | Sodium Laureth-8 Sulfate, | | |
| | Magnesium Laureth Sulfate, | | |
| | Magnesium Laureth-8 Sulfate, | | |
| | Sodium Oleth Sulfate, | | |
| | Magnesium Oleth Sulfate | | |
| Bronidox L | Propylene Glycol, 5-Bromo-5- | 0.20% | (3) |
| | Nitro-1,3-Dioxane | | |
| Everest 79658 SB | Parfum | 0.05% | (4) |
| perfume oil | | | |
| 1% FD&C Blue No. 1 | Aqua (Water), Cl 42090 | 0.20% | (5) |
| in water | (FD&C Blue No. 1) | | |

Phase C

| | | | |
|---|---|---|---|
| Citric acid monohydrate | Citric Acid | 0.15% | (1) |
| Water, demineralised | Aqua (Water) | 10.00% | |

Preparation:

For phase A, the pigment according to the invention is stirred into water. Slowly scatter in Keltrol T with stirring and stir until it has dissolved. Add phases B and C successively while stirring slowly until everything is homogeneously distributed. Adjust the pH to from 6.0 to 6.5.

Sources of Supply:

(1) Merck KGaA/Rona®
(2) Kelco
(3) Cognis GmbH
(4) Haarmann & Reimer GmbH
(5) BASF AG

### Example C

### Eye Shadow

Phase A

| | | | |
|---|---|---|---|
| Pigment | | 55.0% | (1) |
| Biron ® B 50 | Cl 77163 (Bismuth Oxychloride) | 3.00% | (1) |
| Colorona ® Dark Blue | Pearlescent Pigment: Mica, | 10.00% | (1) |
| | Cl 77891 (Titanium Dioxide), | | |
| | Cl 77510 (Ferric Ferrocyanide) | | |
| Magnesium stearate | Magnesium Stearate | 2.50% | (1) |
| White clay (deleted) | Kaolin | 5.00% | (1) |
| Hubersorb 600 | Calcium Silicate | 0.50% | (2) |
| Talc | Talc | 11.00% | (1) |

Phase B

| | | | |
|---|---|---|---|
| Amerchol L 101 | Lanolin Alcohol, Paraffinum | 10.70% | (3) |
| | Liquidum (Mineral Oil) | | |
| Super Hartolan | Lanolin Alcohol | 1.00% | (4) |
| Ewalin 1751 | Petrolatum | 1.00% | (5) |
| Propyl 4-hydroxy- | Propylparaben | 0.10% | (1) |
| benzoate | | | |
| Elegance # 79228 | Parfum | 0.20% | (6) |
| D MD perfume oil | | | |

Preparation:

Combine and pre-mix the constituents of phase A. Subsequently add the molten phase B dropwise to the powder mixture with stirring. The powders are pressed at 40-50 bar.

Sources of Supply:

(1) Merck KGaA/Rona®
(2) J. M. Huber Corp.
(3) Amerchol
(4) Croda GmbH
(5) H. Erhard Wagner GmbH
(6) Haarmann & Reimer GmbH

### Example D

### Eye Shadow Gel

Phase A

| | | | |
|---|---|---|---|
| Pigment | | 15.00% | (1) |
| Mica black | Cl 77499 (Iron Oxides), Mica, | 5.00% | (1) |
| | Cl 77891 (Titanium Dioxide) | | |
| Ronasphere ® | Silica | 3.00% | (1) |
| Carbopol ETD 2001 | Carbomer | 3.00% | (2) |
| Water, demineralised | Aqua (Water) | 60.00% | |

Phase B

| | | | |
|---|---|---|---|
| Glycerol, anhydrous | Glycerin | 2.00% | (1) |
| Germaben II | Propylene Glycol, | 0.20% | (3) |
| | Diazolidinyl Urea, | | |
| | Methylparaben, | | |
| | Propylparaben | | |
| Triethanolamine, extra pure | Triethanolamine | 0.70% | (1) |
| Water, demineralised | Aqua (Water) | 13.80% | |

Preparation:

Pigment and Ronasphere® are dispersed in the water of phase A. Acidify using a few drops of citric acid in order to reduce the viscosity, and scatter in the Carbopol with stirring. After complete dissolution, slowly stir in the pre-dissolved phase B.

Sources of Supply:

(1) Merck KGaA
(2) BF Goodrich GmbH
(3) ISP Global Technologies

### Example E

### Lip Gloss

Phase A

| | | |
|---|---|---|
| Pigment | 10.00% | (1) |

Phase B

| | | | |
|---|---|---|---|
| Indopol H 100 | Polybutene | 59.90% | (2) |
| Bentone MIO V gel | Quaternium-18 Hectorite, | 20.00% | (3) |
| | Propylene Carbonate, | | |
| | Paraffinum Liquidum | | |
| | (Mineral Oil) | | |
| Eutanol G | Octyldodecanol | 6.00% | (4) |
| RonaCareTM tocopherol | Tocopheryl Acetate | 1.00% | (1) |
| acetate | | | |
| Dow Corning 1403 fluid | Dimethiconol, | 3.00% | (5) |
| | Dimethicone | | |
| Propyl 4-hydroxybenzoate | Propylparaben | 0.05% | (1) |
| Covapate W 3773 rouge | Ricinus Communis | 0.05% | (6) |
| | (Castor Oil), Cl 15850 | | |
| | (D&C Red No. 6) | | |

Preparation:

All constituents of phase B are weighed out together, heated to 70° C. and stirred well until a homogeneous composition has formed. The pigments are then added, and the mixture is again stirred. The homogeneous mixture is packaged at 50-60° C.

Sources of Supply:

(1) Merck KGaA/Rona®
(2) BP Amoco
(3) Elementis Specialites
(4) Cognis GmbH
(5) Dow Corning
(6) Les Colorants Wackherr

### Example F

### Eye Shadow—Compact Powder

Phase A

| | | | |
|---|---|---|---|
| Pigment | | 25.00% | (1) |
| Colorona ® Dark Blue | Mica, Cl 77891 (Titanium | 5.00% | (1) |
| | Dioxide), Cl 77510 (Ferric | | |
| | Ferrocyanide) | | |
| Talc | Talc | 49.50% | (1) |
| Potato starch | Solanium Tuberosum | 7.50% | (2) |
| | (Potato Starch) | | |
| Magnesium stearate | Magnesium Stearate | 2.50% | (1) |

Phase B

| | | | |
|---|---|---|---|
| Isopropyl stearate | Isopropyl Stearate | 9.14% | (3) |
| Cetyl palmitate | Cetyl Palmitate | 0.53% | (1) |
| Ewalin 1751 | Petrolatum | 0.20% | (4) |
| Elegance # 79228 D MF | Parfum | 0.20% | (5) |
| perfume oil | | | |
| Propyl 4-hydroxybenzoate | Propylparaben | 0.10% | (1) |

Preparation:

Combine and pre-mix the constituents of phase A. Subsequently add the molten phase B dropwise to the powder mixture with stirring. The powders are pressed at 40-50 bar.

Sources of Supply:

(1) Merck KGaA
(2) Südstärke GmbH
(3) Cognis GmbH
(4) H. Erhard Wagner GmbH
(5) Haarmann & Reimer GmbH

### Example G

### Cream Mascara (O/W)

Phase A

| | | | |
|---|---|---|---|
| Mica black | CI 77499 (Iron Oxides), Mica, | 10.00% | (1) |
| | CI 77891 (Titanium Dioxide) | | |
| Pigment | | 5.00% | (1) |

Phase B

| | | | |
|---|---|---|---|
| Stearic acid | Stearic Acid | 8.00% | (1) |
| Beeswax, bleached | Cera Alba (Beeswax) | 6.00% | (1) |
| Carnauba wax 2442 L | Copernicia Cerifera | 4.00% | (2) |
| | (Carnauba Wax) | | |
| Eutanol G | Octyldodecanol | 3.00% | (3) |
| Arlacel 83 V | Sorbitan Sesquioleate | 2.00% | (4) |
| Propyl 4-hydroxybenzoate | Propylparaben | 0.10% | (1) |
| RonaCareTM tocopherol | Tocopheryl Acetate | 0.50% | (1) |
| acetate | | | |

Phase C

| | | | |
|---|---|---|---|
| Water, demineralised | Aqua (Water) | 50.84% | |
| Triethanolamine, extra pure | Triethanolamine | 2.30% | (1) |
| Water-soluble shellac | Shellac | 8.00% | (5) |
| SSB 63 | | | |
| Methyl 4-hydroxybenzoate | Methylparaben | 0.25% | (1) |
| RonaCareTM biotin | Biotin | 0.01% | (1) |

Preparation:

Melt all constituents of phase B together at about 80° C. and stir until everything has melted. Stir in the diffractive colorants of phase A. Dissolve the shellac of phase C in water and warm to 75° C. Add the remaining constituents of phase C and dissolve. At 75° C., slowly add phase C to phase A/B with stirring and homogenise for 2 min. Cool the composition to room temperature with stirring.

Sources of Supply:

(1) Merck KGaA/Rona®
(2) Kahl & Co.
(3) Cognis GmbH
(4) Uniqema
(5) Paroxite Ltd.

### Example H

### Nail Varnish

Phase A

| | | | |
|---|---|---|---|
| Pigment | | 2.00% | (1) |
| Thixotropic nail varnish | Toluene, Ethyl Acetate, Butyl | 98.00% | (2) |
| base 1348 | Acetate, Nitrocellulose, | | |
| | Tosylamide/Formaldehyde | | |
| | Resin, Dibutyl Phthalate, | | |
| | Isopropyl Alcohol, | | |
| | Stearalkonium Hectorite, | | |
| | Camphor, Acrylates | | |
| | Copolymer, Benzophenone-1 | | |

Colour dispersion with nitrocellulose lacquer (q.s.)
Preparation:

The pigment according to the invention is weighed out together with the varnish base, mixed well by hand using a spatula and subsequently stirred at 1000 rpm for 10 min.

Sources of Supply:

(1) Merck KGaA/Rona®
(2) International Lacquers S. A.

### Example J

### Shampoo

Phase A

| | | | |
|---|---|---|---|
| Pigment | | 0.05% | (1) |
| Carbopol ETD 2020 | Acrylates/C10-30 Alkyl Acrylate | 0.90% | (2) |
| | Crosspolymer | | |
| Water, demineralised | Aqua (Water) | 59.90% | |

Phase B

| | | | |
|---|---|---|---|
| Triethanolamine, | Triethanolamine | 0.90% | (1) |
| extra pure | | | |
| Water, demineralised | Aqua (Water) | 10.00% | |

Phase C

| | | | |
|---|---|---|---|
| Plantacare 2000 UP | Decyl Glucoside | 20.00% | (3) |
| Texapon ASV | Magnesium Oleth Sulfate, | 8.00% | (3) |
| | Sodium Oleth Sulfate, | | |
| | Magnesium Laureth-8 Sulfate, | | |
| | Sodium Laureth-8 Sulfate, | | |
| | Magnesium Laureth Sulfate, | | |
| | Sodium Laureth Sulfate | | |
| Bronidox L | Propylene Glycol, 5-Bromo- | 0.20% | (3) |
| | 5-Nitro-1,3-Dioxane | | |
| Everest 79658 SB | Parfum | 0.05% | (4) |
| perfume oil | | | |

Preparation:

For phase A, stir the pigment according to the invention into the water.

Acidify using a few drops of citric acid (10%) in order to reduce the viscosity, and slowly scatter in the Carbopol with stirring. After complete dissolution, slowly add phase B. The constituents of phase C are then added successively.

Sources of Supply:

(1) Merck KGaA
(2) BF Goodrich GmbH
(3) Cognis GmbH
(4) Haarmann & Reimer GmbH

### Example K

### Artificial Nail with Opal Effect

A film is produced as described in one of Examples 7, 9, 11, 11a or 14c without the grinding step described and brought into a shape which is suitable as artificial nail or as coating for a nail.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

The entire disclosure of all applications, patents and publications, cited herein and of corresponding German application No. 10 2004 032 120.5, filed Jul. 1, 2004 is incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Use of diffractive colorants in cosmetics.

2. Use according to claim 1, characterised in that the diffractive colorants are employed for decorative purposes in topical application.

3. Use according to claim 1, characterised in that the diffractive colorants are employed for decorative purposes in compositions for topical application.

4. Use according to claim 1, characterised in that the diffractive colorants are particles, films or aggregates.

5. Use according to claim 1, characterised in that the diffractive colorants are employed in nail cosmetics.

6. Use according to claim 1, characterised in that the diffractive colorants essentially consist, in the proportion essential for the optical properties, of arrangements of spherical particles or cavities having an essentially monodisperse size distribution.

7. Use according to claim 6, characterised in that the spherical particles are embedded in a wax-like matrix.

8. Use according to claim 1, characterised in that the spherical particles essentially consist of an organic polymer, which is preferably crosslinked.

9. Use according to claim 1, characterised in that the spherical particles essentially consist of an inorganic material, preferably a metal or semimetal or a metal chalcogenide or metal pnictide, particular preference being given to silicon dioxide, aluminium oxide, zirconium oxide, iron oxides, titanium dioxide, cerium dioxide, gallium nitride, boron nitride, aluminium nitride, silicon nitride and phosphorus nitride or mixtures thereof, as core material.

10. Use according to claim 1, characterised in that the spherical particles essentially consist of polymers which include particles, for example metal oxides.

11. Use according to claim 1, characterised in that the diffractive colorants essentially consist, in the proportion essential for the optical properties, of core/shell particles whose shell forms a matrix and whose core is essentially solid and has an essentially monodisperse size distribution, where there is a difference between the refractive indices of the core material and of the shell material.

12. Use according to claim 11, characterised in that the matrix is brittle, and the shell material is preferably a homo- or copolymeric poly(cyclohexyl methacrylate), polystyrene or substituted polystyrene derivative, such as, for example, poly(iodostyrene) or poly(bromostyrene), polyacrylate or polymethacrylate having a Tg above the service temperature, polyvinyl chloride having a high Tg or another vinylic polymer obtained by conversion from polyvinyl acetate, or is polyacrylonitrile or styrene-acrylonitrile copolymer.

13. Use according to claim 1, characterised in that the diffractive colorants have cavities, and the matrix around the cavities essentially consists of an organic polymer, which is preferably cross-linked.

14. Use according to claim 1, characterised in that the diffractive colorants have cavities, and the matrix around the cavities essentially consists of an inorganic material, preferably a metal or semimetal or a metal chalcogenide or metal pnictide, particular preference being given to silicon dioxide, aluminium oxide, zirconium oxide, iron oxides, titanium dioxide, cerium dioxide, gallium nitride, boron nitride, aluminium nitride, silicon nitride and phosphorus nitride, or mixtures thereof.

15. Use according to claim 1, characterised in that the matrix has thermoplastic or thermosetting properties and is preferably built up from poly(styrene), thermoplastic poly(acrylate) derivatives, preferably poly(methyl methacrylate) or poly(cyclohexyl methacrylate), or thermoplastic copolymers of these polymers with other acrylates, such as, preferably, styrene-acrylonitrile copolymers, styrene-ethyl acrylate copolymers or methyl methacrylate-ethyl acrylate copolymers.

16. Use according to claim 1, characterised in that the diffractive colorants have cavities, and the matrix is a carbon matrix.

17. Use according to claim 1, characterised in that the average diameter of the spherical particles or cavities in the diffractive colorants is in the range of about 50-500 nm, preferably in the range of 100-500 nm and very particularly preferably in the range from 200 to 280 nm.

18. Composition for topical application comprising at least one diffractive colorant and at least one carrier which is suitable for topical application.

19. Composition according to claim 18, characterised in that the total amount of the diffractive colorant(s) is selected from the range from 0.1% by weight to 30% by weight, based on the total weight of the composition.

20. Composition according to claim 1, characterised in that the composition comprises at least one dye or pigment, where the composition preferably comprises at least one effect pigment, in particular a pearlescent pigment.

21. Composition according to claim 1, characterised in that the composition comprises at least one substance which absorbs UV radiation in the UV-A and/or UV-B range, where the at least one substance is preferably selected from 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazole-5-sulfonic acid and the potassium, sodium and triethanolamine salts thereof.

22. Process for the preparation of a composition for topical application, characterised in that at least one diffractive colorant is mixed with at least one carrier which is suitable for topical application and optionally further ingredients.

23. Process for assessment of the application of compositions for topical application, characterised in that diffractive colorants which are incorporated into the composition are applied to the skin, and it is estimated from the visible effect of the diffractive colorants to which parts of the skin application has already taken place and/or whether sufficient composition has been applied and/or when re-application has become necessary.

24. Diffractive colorant, characterised in that it is an aggregate comprising arrangements of spherical particles having an essentially monodisperse size distribution in a wax-like matrix.

25. Diffractive colorant according to claim 24, characterised in that the wax-like matrix is selected from fats, waxes and other natural and synthetic fatty bodies, preferably esters of fatty acids with alcohols having a low C number, for example with isopropanol, propylene glycol or glycerol, or esters of fatty alcohols with alkanoic acids having a low C number or with fatty acids, where the wax-like matrix is preferably selected from cetyl palmitate and lanolin.

26. Process for the preparation of a diffractive colorant, in which, in a first step, three-dimensional sphere packing is produced, and, in a second step, the sphere aggregates are impregnated with a melt of a material which is wax-like at room temperature.

27. Artificial nail comprising diffractive colorants.

28. Nail-care composition comprising solvents and binders, characterised in that the nail-care composition comprises diffractive colorants or precursors of diffractive colorants, in particular in the form of monodisperse spheres or core/shell particles.

29. Nail-care composition according to claim 28, characterised in that it is a water-based nail varnish which preferably comprises polyurethanes or polyacrylates as binder.

30. Nail-care composition according to claim 28, characterised in that the diffractive colorant or the precursor of the diffractive colorant simultaneously serves as binder, where the nail-care composition preferably comprises no further binder, and the precursor of the diffractive colorant is preferably core/shell particles, preferably having a polyacrylate shell.
